(19) United States
(12) Patent Application Publication
(10) Pub. No.: US 2017/0056621 A1
(43) Pub. Date: Mar. 2, 2017

(54) LOCAL ADMINISTRATION OF DRUGS FOR THE TREATMENT OF ASTHMA

(71) Applicant: Mercator MedSystems, Inc., Emeryville, CA (US)

(72) Inventors: Emily STEIN, San Leandro, CA (US); Kirk Patrick SEWARD, San Francisco, CA (US)

(21) Appl. No.: 15/252,683

(22) Filed: Aug. 31, 2016

Related U.S. Application Data
(60) Provisional application No. 62/212,330, filed on Aug. 31, 2015.
(60) Provisional application No. 62/267,666, filed on Dec. 15, 2015.

(51) Int. Cl. A61M 25/01; A61K 38/57; A61K 31/573

(52) U.S. Cl. CPC A61M 25/01; A61K 31/573; A61K 38/57; A61M 2025/1052

(57) ABSTRACT

Methods for treating inflammatory pulmonary diseases by administering to the patient an effective amount of one or more pharmaceutical agents are disclosed. The pharmaceutical agents administered can include antibiotics, steroids, NSAIDs, DMARDs, growth factor receptor inhibitors, PI3K inhibitors, neurotransmitter receptor inhibitors, or protease inhibitors. The dose administered is effective to suppress or prevent initiation, progression, or relapses of disease, including the progression of established disease. The pharmaceutical agent is administered to a patient determined to have the disease and at an amount effective to suppress or prevent activity of the disease. The pharmaceutical agent is administered using a transtracheal, transbronchial, or transvascular drug delivery catheter. The pharmaceutical agent can be administered to the patient's pulmonary tissue to suppress reactions in response to bronchial thermoplasty either before, during, or after bronchial thermoplasty.

### CROSS-REFERENCE

This application claims the benefit of U.S. Provisional Applications No. 62/212,330, filed on Aug. 31, 2015, and 62/267,666, filed on Dec. 15, 2015, the entire contents of which are incorporated herein by reference.

### BACKGROUND OF THE INVENTION

1. Field of the Invention

There is a long-standing interest in the development of improved treatments for asthma, which affects over 300 million people worldwide, with an estimated 17.4% of patients' disease considered difficult-to-control and 3.6% of patients demonstrating severe refractory disease. Despite the availability of effective therapies in the market, an increasing number of patients are becoming refractory or have suboptimal asthma control, requiring the development of novel treatment paradigms to address these disparities in asthma.

Airway inflammation including bronchial hyperresponsiveness and airway remodeling are predominant features of asthma, a phenotypically heterogeneous chronic respiratory disease. Significant evidence points to a role for aberrant bronchial epithelial cell and immune cell activity in classic asthma, characterized by eosinophilic infiltrate, T helper 2 (Th2) and Th9 lymphocyte development, and release of cytokines such as IL5, IL4, IL9 and IL13.

Emerging evidence also points to differential neutrophilic infiltrate, Th1 and Th17 lymphocyte skewing and release of cytokines such as IL1, IL8, IFN, and IL17/IL23 in severe asthma. Taken together, these suggest a role for dendritic cell-mediated T lymphocyte subset polarization, likely dependent on the particular airway milieu in a given patient.

Current approaches to treat asthma and other inflammatory pulmonary diseases are categorized into two general classes, long-term control medications to achieve and maintain control of persistent disease, and quick-relief medications for treating acute symptoms and exacerbations, most requiring passive drug uptake by target cells through oral or aerosolized delivery. While effective in many patients, a growing number of patients are refractory to current approaches, requiring more improved treatment strategies for controlling disease. A recent approach of bronchial thermoplasty, applying radiofrequency energy to the airway in severe asthma patients, is also used clinically but elicits inflammation at the targeted sites and is associated with spasm and severe asthma exacerbations.

Novel methods of administration and uses of existing drugs for the treatment of asthma and adjunctive therapy with bronchial thermoplasty are provided herein.

2. Description of the Background Art

U.S. Pat. No. 6,547,803 B2, and published patent Applications US 2003/0171734 A1, US 2014/0107478 A1, and US 2014/0303569 A1 describe microneedle catheters which may be used in at least some of the methods described in the present application, and are hereby incorporated by reference in their entirety.

### SUMMARY

The present disclosure generally relates to medical devices and methods. More particularly, the present disclosure relates to medical devices and methods for distributing pharmaceutical agents to pulmonary tissue for the treatment of asthma, COPD, or other inflammatory pulmonary disease.

Aspects of the present disclosure provide methods for inhibiting cellular and molecular drivers of chronic asthma in mammals with an effective dose of a pharmaceutical agent administered to the pulmonary tissue such as by a micro-needle catheter and in a manner that can bypass the pulmonary mucosal epithelial layer for improved pharmaceutical agent uptake and efficacy. In some embodiments, the pharmaceutical agent is administered as an adjunctive therapy with bronchial thermoplasty. In some embodiments, the pharmaceutical agent is an effective dose of one or more antibiotics. In other embodiments, the pharmaceutical agent is an effective dose of one or more of a smooth muscle relaxant, non-steroidal anti-inflammatory, anti-cytokine antibody, steroid, EGFR inhibitor, PDGFR inhibitor, PI3K inhibitor, neurotransmitter receptor inhibitor or protease inhibitor, or the like. Further aspects of the present disclosure provide methods for the transbronchial or transtracheal administration of a bolus of pharmaceutical agent directly into pulmonary tissue for the treatment of asthma, COPD, or other inflammatory pulmonary disease. Further aspects of the present disclosure provide methods for the transvascular administration of a bolus of pharmaceutical agent directly into pulmonary tissue for the treatment of asthma, COPD, or other inflammatory pulmonary disease. Further aspects of the present disclosure provide methods for the transvascular, transbronchial, or transtracheal administration of a bolus of pharmaceutical agent directly into pulmonary tissue for the treatment of asthma, COPD, or other inflammatory pulmonary disease prior to, at the time of, or after bronchial thermoplasty. Following administration of the pharmaceutical agent, the inhibition of drugged target activity or disease manifestations may be determined.

Asthma-related pharmaceutical agents disclosed herein include at least those of members of the antibiotic, vasodilator, non-steroidal anti-inflammatory (NSAID), steroid, anti-cytokine antibody, smooth muscle relaxant, EGFR inhibitor, PDGFR inhibitor, FGFR inhibitor, PI3K inhibitor, goblet cell antagonist, immune-related neurotransmitter receptor inhibitor or protease inhibitor classes, and include naturally occurring and synthetic compounds.

In some embodiments, a mammalian host suffering from asthmatic disease having undesirable activity of resident microorganism, immune cell, mucosal epithelial cell, smooth muscle cell, or goblet cell or effector molecules produced by said cell types in pulmonary tissue or the like can be treated with an effective dose of one or more pharmaceutical agents. The methods disclosed may further comprise administering to the host an effective amount of one or more pharmaceutical agents by an intravascular catheter, intrabronchial catheter, or intratracheal catheter, where the dose may be effective to suppress or prevent initiation, progression, or relapses of disease, including the progression of established disease. In some embodiments, the methods disclosed comprise administering to a patient having pre-existing inflammatory pulmonary symptoms, an effective amount of one or more of an antibiotic, vasodilator, non-steroidal anti-inflammatory (NSAID), steroid, smooth muscle relaxant, anti-cytokine antibody, growth factor inhibitor, PI3K inhibitor, or protease inhibitor, or the like to suppress or prevent relapses of the disease. In some embodiments, the pharmaceutical agent may be administered in a single bolus. In other embodiments, the pharmaceutical agent may be administered in a series of injections to provide therapeutic relief. In some embodiments, a patient may be selected if he or she has an inflammatory pulmonary disease, e.g., asthma, by a suitable diagnostic method, prior to administration of a therapeutic dose of the pharmaceutical agent. In some embodiments, the inflammation, e.g., secretion of cytokines, bronchial spasm, hyper-secretion or aberrant accumulation of mucus, hyper-proliferation, tissue remodeling, and the like, may be determined prior to and following said administration. In other embodiments, the patient immune response may be monitored prior to and following administration of the pharmaceutical agent. Yet in other embodiments, in a patient receiving bronchial thermoplasty, a pharmaceutical agent may be administered as a bolus, as a series of injections or administered on an as-needed basis to provide relief from disease symptoms and/or undesirable effects of bronchial thermoplasty. In yet other embodiments, a patient may require administration of the pharmaceutical agent prior to bronchial thermoplasty to reduce occurrence of asthma exacerbations or local inflammation, swelling, and bronchial obstruction or narrowing.

In some embodiments, a pharmaceutical agent may be combined with one or more pharmaceutical agents, where the combination may provide for a synergistic effect. The combination may allow for use of a reduced dose of one or both agents. In some embodiments, the one or more agents may inhibit pro-asthmatic signaling. In other embodiments, the one or more agents may be a steroid. In other embodiments, the one or more agents may be a disease modifying anti-rheumatic agent.

Aspects of the present disclosure provide methods for inhibiting an inflammatory pulmonary disease in a patient. An exemplary method may comprise steps of advancing a delivery catheter through a bodily lumen of the patient to a position adjacent a target site in pulmonary tissue, advancing a delivery needle laterally from a lateral side of the delivery catheter through a wall of the bodily lumen to access the target site, and injecting a therapeutically effective dose of a pharmaceutical agent to the target site.

In some embodiments, the therapeutically effective dose of the pharmaceutical agent is effective to suppress or prevent initiation, progression, or relapses of disease, including the progression of established disease.

In some embodiments, advancing the delivery catheter through the bodily lumen comprises advancing the delivery catheter through a blood vessel, and advancing the delivery needle laterally from the delivery catheter through the wall of the bodily lumen comprises advancing the delivery needle through a wall of the blood vessel.

In some embodiments, advancing the delivery catheter through the bodily lumen comprises advancing the delivery catheter through a trachea, and advancing the delivery needle laterally from the delivery catheter through the wall of the bodily lumen comprises advancing the delivery needle into or through a wall of the trachea.

In some embodiments, advancing the delivery catheter through the bodily lumen comprises advancing the delivery catheter through a bronchus or bronchi, and advancing the delivery needle laterally from the lateral side of the delivery catheter into or through the wall of the bodily lumen comprises advancing the delivery needle into or through a wall of the bronchus or bronchi.

In some embodiments, advancing the delivery needle laterally from the delivery catheter comprises expanding an expandable element disposed on a distal portion of the catheter to extend the delivery needle laterally from the expandable element, thereby placing a section of the expandable element adjacent the delivery needle in contact with the wall of the bodily lumen.

In some embodiments, the section of the expandable element adjacent the delivery needle in contact with the wall of the bodily lumen seals and prevents leakage of the pharmaceutical agent delivered from the laterally extended delivery needle back into the bodily lumen. Further, in some embodiments one or more sections of the expandable element adjacent one or more delivery needles are in contact with the wall of the bodily lumen to seal and prevent leakages of the pharmaceutical agent delivered from the one or more laterally extended delivery needles back into the bodily lumen.

In some embodiments, the section of the expandable element adjacent the delivery needle in contact with the wall of the bodily lumen seals a tissue tract of the laterally extended delivery needle. Further, in some embodiments one or more sections of the expandable element adjacent one or more delivery needles are in contact with the wall of the bodily lumen to seal one or more tissue tracts of the one or more laterally extended delivery needles.

In some embodiments, the inflammatory pulmonary disease comprises asthma, COPD, or infection.

In some embodiments, the method for inhibiting an inflammatory pulmonary disease in a patient comprises diagnosing the patient as having the inflammatory pulmonary disease prior to injecting the therapeutically effective dose of the pharmaceutical agent.

In some embodiments, the method for inhibiting an inflammatory pulmonary disease in a patient comprises monitoring the status of the patient affected by the pulmonary inflammatory disease following injecting the therapeutically effective dose of the pharmaceutical agent.

In some embodiments, monitoring the status of the patient affected by the pulmonary inflammatory disease comprises monitoring pulmonary tissues by MRI, x-ray, CT, spirometry, PCR, ELISA, NGS, or culture.

In some embodiments, the pharmaceutical agent is administered in combination with one or more pharmaceutical agents. Further, in some embodiments the pharmaceutical agent is administered in a composition that includes various other agents to enhance delivery and efficacy, and with active and inactive compounds.

In some embodiments, the therapeutically effective dose of the pharmaceutical agent is injected prior to, during, or following bronchial thermoplasty.

In some embodiments the pharmaceutical agent comprises one or more of alpha-1-antitrypsin, tofacitinib, scopolamine, ceftriaxone, anti-IL5 antibody, anti-IL13 antibody, anti-33 antibody, prednisolone, or dexamethasone. In some embodiments, the pharmaceutical agent comprises one or more of an antibiotic, DMARD, steroid, NSAID, smooth muscle relaxant, EGFR antagonist, PDGFR antagonist, PI3K inhibitor, neurotransmitter receptor inhibitor, growth factor receptor inhibitor, or protease inhibitor. In some embodiments, the pharmaceutical agent comprises a short-acting beta agonist (SABA) such as albuterol, levalbuterol or pirbuterol. In some embodiments, the pharmaceutical agent comprises a smooth muscle relaxant (SMR) such tiotropium bromide, theophylline, hydralazine, clenbuterol, flavoxate, dicycloverine, papaverine, hyoscine hydrobromide, carisoprodol, cyclobenzaprine, metataxalone, methocarbamol, tizanidine, diazepam, baclofen, a substance P inhibitor, dantrolene, chlorzoxazone, gabapentin, or orphenadrine.

Aspects of the present disclosure provide pharmaceutical agents for use in a method of inhibiting an inflammatory pulmonary disease. An exemplary pharmaceutical agent may be for delivery to a target site in pulmonary tissue, such as by micro-needle catheter, bypassing the pulmonary mucosal epithelial layer. The pharmaceutical agent may suppress or prevent initiation, progression, or relapses of the disease, including the progression of established disease.

In some embodiments, the pharmaceutical agent is for delivery by a pre-situated micro-needle catheter that has previously been advanced through a bodily lumen to a position adjacent to the target site, and the micro-needle for delivery is extended laterally from a lateral side of the catheter through a wall of the bodily lumen to access the target site prior to the delivery of the pharmaceutical agent. The bodily lumen may comprise a blood vessel, a trachea, or a bronchus, and the micro-needle for delivery may be extended laterally from the lateral side of the catheter through a wall of the blood vessel, a wall of the trachea, or a wall of the bronchus, respectively, to access the target site.

In some embodiments, the micro-needle is extended laterally from the lateral side of the catheter prior to delivery of the pharmaceutical agent by expanding an expandable element disposed on a distal end of the catheter to extend the needle laterally from the expandable element, thereby placing a section of the expandable element adjacent the needle in contact with a wall of the lumen. The section of the expandable element adjacent the needle in contact with the wall of the lumen may prevent leakage of the pharmaceutical agent from the laterally extended needle back into the lumen. Alternatively or in combination, extension of the needle through the wall of the bodily lumen may generate a tissue tract, the section of the expandable element adjacent to the needle in contact with the wall of the lumen sealing the tissue tract from the bodily lumen.

In some embodiments, the inflammatory pulmonary disease is asthma, COPD or infection.

In some embodiments, a patient to be treated is diagnosed as having the inflammatory pulmonary disease prior to delivery of the pharmaceutical agent.

In some embodiments, the status of a patient affected by the pulmonary inflammatory disease is monitored following delivery of the pharmaceutical agent. The monitoring may be by MRI, x-ray, CT, spirometry, PCR, ELISA, NGS, or culture, to name a few examples.

In some embodiments, the pharmaceutical agent is administered in combination with one or more additional pharmaceutical agent.

In some embodiments, the pharmaceutical agent is delivered prior to, during, or following bronchial thermoplasty.

In some embodiments, the pharmaceutical agent comprises one or more of an antibiotic, DMARD, steroid, NSAID, smooth muscle relaxant, EGFR antagonist, PDGFR antagonist, PI3K inhibitor, neurotransmitter receptor inhibitor, growth factor receptor inhibitor, or protease inhibitor.

In some embodiments, the pharmaceutical agent comprises one or more of alpha-1-antitrypsin, tofacitinib, scopolamine, ceftriaxone, anti-IL5 antibody, anti-IL13 antibody, anti-33 antibody, prednisolone, or dexamethasone.

In some embodiments, the pharmaceutical agent comprises one or more of albuterol, levalbuterol or pirbuterol.

In some embodiments, the pharmaceutical agent comprises one or more of tiotropium bromide, theophylline, hydralazine, clenbuterol, flavoxate, dicycloverine, papaverine, hyoscine hydrobromide, carisoprodol, cyclobenzaprine, metataxalone, methocarbamol, tizanidine, diazepam, baclofen, a substance P inhibitor, dantrolene, chlorzoxazone, gabapentin, or orphenadrine.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 is a cross-sectional illustration of a bronchial lumen with surrounding tissue illustrating the relationship between the lumen and bronchial lumen wall components;

FIG. 1A is a perspective view of an exemplary microfabricated surgical device for interventional procedures in an unactuated condition;

FIG. 1B is a side sectional view along line 1B-1B of FIG. 1A;

FIG. 1C is a side sectional view along line 1C-1C of FIG. 1A;

FIG. 1D is a cross-sectional illustration of a microneedle of an exemplary microfabricated surgical device for interventional procedures having a pharmaceutical agent delivery aperture positioned beyond the pulmonary mucosal epithelium (E);

FIG. 1E is a cross-sectional illustration of the volumetric drug distribution achieved by the microneedle positioning of FIG. 1D;

FIG. 2A is a perspective view of the exemplary microfabricated surgical device of FIG. 1A in an actuated condition;

FIG. 2B is a side sectional view along line 2B-2B of FIG. 2A;

FIG. 3 is a side view of an exemplary microfabricated surgical device for interventional procedures inserted into a lumen of a patient;

FIGS. 3A, 3B, and 3C are cross-sectional views illustrating the injection of a radio contrast media to help determine whether the pharmaceutical agent delivery aperture of a microneedle of an exemplary microfabricated surgical device for interventional procedures is properly placed within the preferred periluminal space surrounding a lumen of a patient;

FIG. 3D is a side view illustrating the optional placement of sensors on an exemplary pharmaceutical agent delivery needle, which sensors can detect whether the needle has been advanced into the preferred periluminal space surrounding a lumen of a patient;

FIG. 4 is a perspective view of another embodiment of a microfabricated surgical device for interventional procedures;

FIG. 5 is a side view of another embodiment of a microfabricated surgical device for interventional procedures, as inserted into a lumen of a patient;

FIGS. 6A and 6B illustrate the initial stage of an injection of a pharmaceutical agent into a periluminal space using the exemplary microfabricated surgical device for interventional procedures of FIG. 3; FIG. 6A is a cross-section and FIG. 6B a longitudinal section taken across an internal lumen of a patient;

FIGS. 7A and 7B are similar to FIGS. 6A and 6B showing the extent of pharmaceutical agent distribution at a later time after injection;

FIGS. 8A and 8B are similar to FIGS. 6A and 6B showing the extent of pharmaceutical agent distribution at a still later time after injection;

FIGS. 9A, 9B, 9C, 9D, and 9E are cross-sectional views of an exemplary fabrication process employed to create a free-standing low-modulus patch within a higher modulus anchor, framework, or substrate;

FIGS. 10A, 10B, 10C, and 10D are cross-sectional views of the inflation process of an exemplary microfabricated surgical device for interventional procedures;

FIGS. 11A, 11B, and 11C are cross-sectional views of an exemplary microfabricated surgical device for interventional procedures, illustrating the ability to treat multiple lumen diameters;

FIG. 12 shows a flow chart of an exemplary method of the treatment of a patient with an inflammatory pulmonary disease, according to many embodiments;

FIG. 13 shows a schematic anterior-view illustration of the gross anatomy of the lung;

FIG. 14A is a schematic anterior view of a patient defining transverse plane 14A-14A to show the vascularization of the lung;

FIG. 14B is a schematic cross-sectional view along transverse plane 14A-14A of FIG. 14A showing the vascularization of the lung;

FIG. 15A is a schematic anterior-view cross-sectional illustration showing an exemplary transtracheal delivery route for diagnostic and/or therapeutic agent delivery to treat a patient with an inflammatory pulmonary disease.

FIG. 15B is a schematic anterior-view cross-sectional illustration showing an exemplary transbronchial delivery route for diagnostic and/or therapeutic agent delivery to treat a patient with an inflammatory pulmonary disease.

### DETAILED DESCRIPTION

Specific embodiments of the disclosed device, delivery system, and method will now be described with reference to the drawings. Nothing in this detailed description is intended to imply that any particular component, feature, or step is essential to the invention.

The present disclosure will preferably utilize microfabricated surgical devices, more specifically microfabricated catheters, for transvascular, transtracheal, or transbronchial injection of one or more pharmaceutical agents into pulmonary tissue. The following description provides representative embodiments and methods of use of catheters having one or more microneedles suitable for the delivery of one or more pharmaceutical agents to the pulmonary tissue of a patient with an inflammatory pulmonary disease. The following description further provides representative pharmaceutical agents for the treatment of an inflammatory pulmonary disease in a patient delivered by the catheters having one or more microneedles described herein. The following description further provides methods for the treatment of an inflammatory pulmonary disease in a patient prior to, during, or following bronchial thermoplasty.

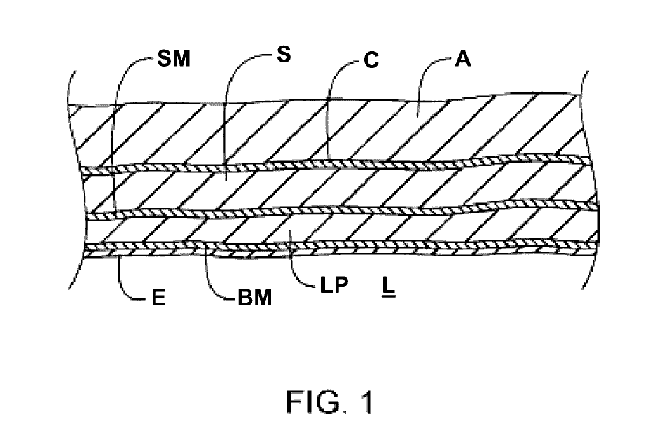

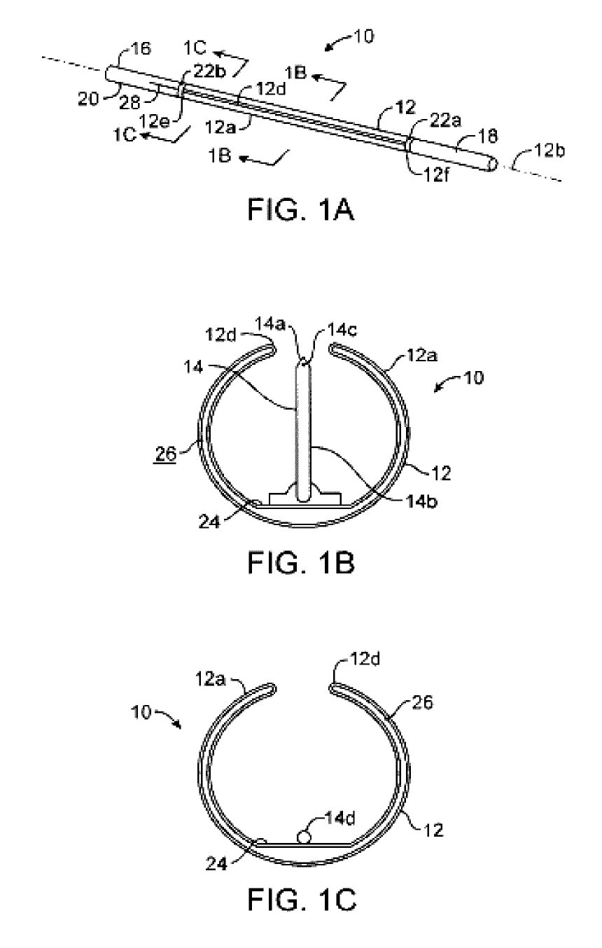

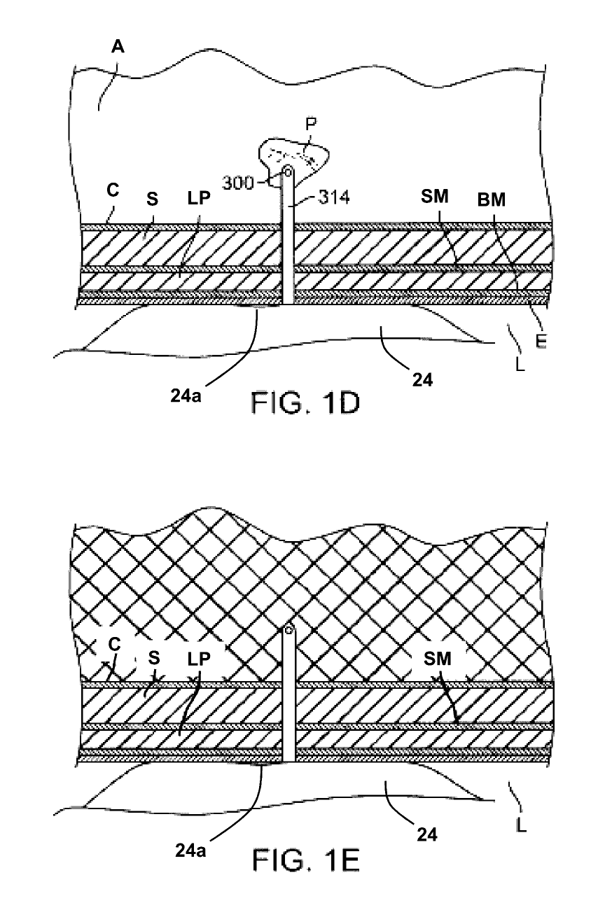

The benefits of the disclosed device, delivery system, and methods are achieved by delivering pharmaceutical agents into a periluminal space surrounding a lumen of a patient, wherein the lumen may comprise any of a bronchus, bronchi, artery, vein, vessel, or the like. By way of example, FIG. 1 shows a typical bronchial wall in cross-section where the pulmonary mucosal epithelium E is the layer of the wall which is exposed to the bronchial lumen L. Underlying the pulmonary mucosal epithelium E is the basement membrane BM which in turn is surrounded by the lamina propia LP. The lamina propia LP, in turn, is surrounded by smooth muscle SM over which is located the submucosa S. As shown in FIG. 1, the submucosa S is in turn surrounded by cartilage plates C, beyond which lies the adventitia A. The cartilage plates C may also be interspersed within the submucosa S and the adventitia A. In this example, the periluminal space can be considered anything lying beyond the pulmonary mucosal epithelium E, including regions within the adventitia A and beyond. The trachea may comprise components similar to the bronchus as described. Related to this example but not shown, the periluminal space can also be defined as the region beyond the external elastic lamina of an artery, or beyond the tunica media of a vein.

For delivery of one or more pharmaceutical agents to a periluminal space, one or more microneedles of a pharmaceutical agent delivery catheter may be inserted, preferably in a substantially normal direction, into the wall of a lumen to eliminate as much trauma to the patient as possible. Until the microneedle is at the site of an injection, it may be positioned out of the way so that it does not scrape against lumen walls with its tip. Specifically, the microneedle may remain enclosed in the walls of an actuator or sheath attached to a catheter so that it will not injure the patient during intervention or the physician during handling. When the injection site is reached, movement of the actuator along the lumen can be terminated, and the actuator may be operated to cause the microneedle to be thrust outwardly, substantially perpendicular to the central axis of a lumen, for instance, in which the catheter has been inserted. The actuator may be in the form of an expandable element located at the distal end of the catheter, and actuating the actuator may include expanding the expandable element. In some embodiments, the injection site may be chosen from sub-epithelial tissue of the bronchus such as its lamina propia LP, its smooth muscle SM, and its adventitia A, may be chosen from sub-epithelial tissue of the trachea, or may be chosen from sub-epithelial tissue of a blood vessel.

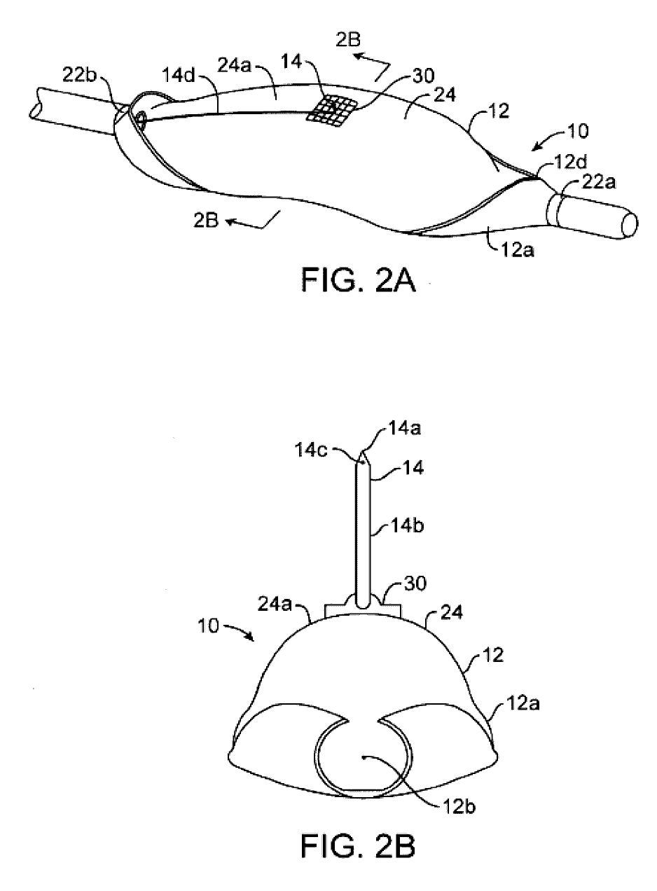

Shown in FIGS. 1A, 1B, 1C, 1D, 1E, 2A, and 2B, is a microfabricated intraluminal catheter 10 including an actuator 12 having an actuator body 12a and central longitudinal axis 12 b. The actuator body can more or less form a C-shaped outline having an opening or slit 12d extending substantially along its length. A microneedle 14 may be located within the actuator body, as discussed in more detail below, when the actuator is in its unactuated condition (furled state) (FIG. 1B). The microneedle can be moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 10. The catheter tip end can serve as a means of locating the actuator inside a lumen of a patient by use of a radiopaque coatings or markers. The catheter tip can also form a seal at the distal end 12f of the actuator. The lead end of the catheter can provide the necessary interconnects (fluidic, mechanical, electrical or optical) at the proximal end 12e of the actuator.

Retaining rings 22a and 22b may be located at or formed into the distal and proximal ends, respectively, of the actuator. The catheter tip may be joined to the retaining ring 22a, while the catheter lead may be joined to retaining ring 22b. The retaining rings can be made of a thin, on the order of 10 to 100 microns (μm), substantially rigid material, such as parylene (types C, D or N), or a metal, for example, aluminum, stainless steel, gold, titanium or tungsten. The retaining rings may form a rigid substantially “C”-shaped structure at each end of the actuator. The catheter may be joined to the retaining rings by, for example, a butt-weld, an ultra-sonic weld, integral polymer encapsulation or an adhesive such as an epoxy.

The actuator body may further comprise a central, expandable section 24 located between retaining rings 22a and 22b. The expandable section 24 may include an interior open area 26 for rapid expansion when an activating fluid is supplied to that area. The central section 24 may be made of a thin, semi-rigid or rigid, expandable material, such as a polymer, for instance, parylene (types C, D or N), silicone, polyurethane or polyimide. The central section 24, upon actuation, may be expandable somewhat like a balloon-device.

The central section may be capable of withstanding pressures of up to about 100 atmospheres upon application of the activating fluid to the open area 26. The material from which the central section is made of may be rigid or semi-rigid in that the central section returns substantially to its original configuration and orientation (the unactuated condition) when the activating fluid is removed from the open area 26. Thus, in this sense, the central section can be very much unlike a balloon which has no inherently stable structure.

The open area 26 of the actuator may be connected to a delivery conduit, tube or fluid pathway 28 that extends from the catheter's lead end to the actuator's proximal end. The activating fluid can be supplied to the open area via the delivery tube. The delivery tube may be constructed of Teflon© or other inert plastics. The activating fluid may be a saline solution or a radio-opaque dye.

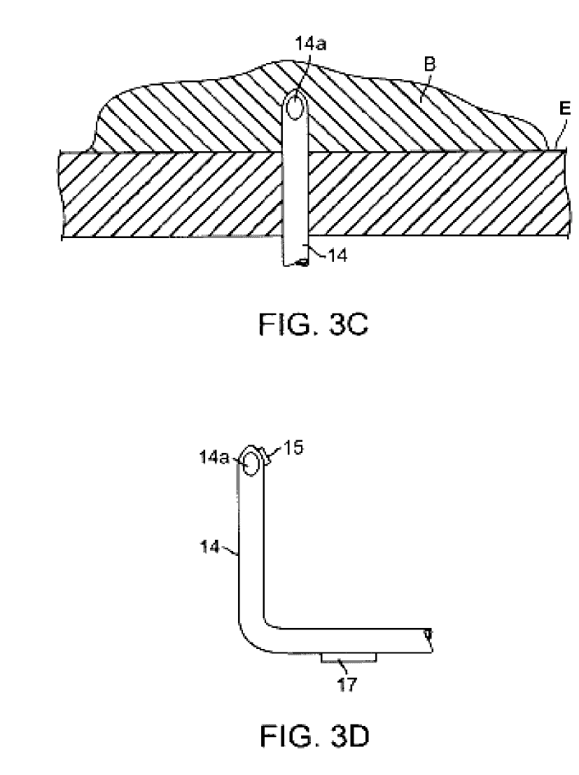

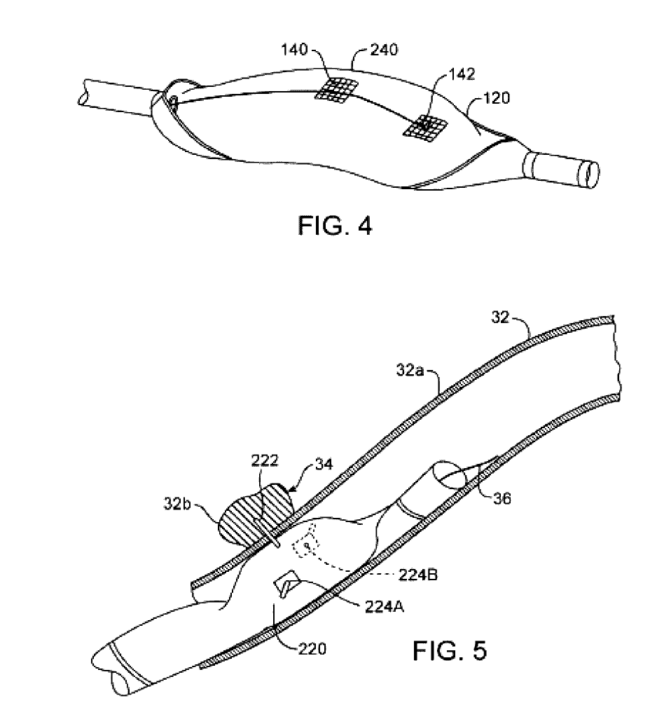

The microneedle 14 may be located approximately in the middle of the central section 24. However, as discussed below, this may not be necessary, especially when multiple microneedles are used. The microneedle may be affixed to an exterior surface 24a of the central section. The microneedle may be affixed to the surface 24a by an adhesive, such as cyanoacrylate. Alternatively or in combination, the microneedle may be joined to the surface 24a by a metallic or polymer mesh-like structure 30 (see FIG. 4), which is itself affixed to the surface 24a by an adhesive. The mesh-like structure may be made of, for instance, steel or nylon.

The microneedle includes a sharp tip 14a and a shaft 14b. The microneedle tip can provide an insertion edge or point. The shaft 14b can be hollow and the tip can have an outlet port 14c, permitting the injection of a pharmaceutical agent into a patient. The microneedle, however, does not need to be hollow, as it may be configured like a neural probe to accomplish other tasks.

As shown, the microneedle can extend approximately perpendicularly from surface 24a. Thus, as described, the microneedle may move substantially perpendicularly to an axis of a lumen into which has been inserted, to allow direct puncture or breach of a lumen wall. The direct puncture or breach of the microneedle of a lumen wall can thus create a tissue tract in the lumen wall.

The microneedle may further include a pharmaceutical or drug supply conduit, tube or fluid pathway 14d which can place the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 34-gauge, 30-gauge, or smaller, steel needle. Alternatively or in combination, the microneedle may be microfabricated from polymers, other metals, metal alloys or semiconductor materials. The needle, for example, may be made of Parylene, silicon or glass. Microneedles and methods of fabrication are described in U.S. patent publication 2002/0188310, entitled “Microfabricated Surgical Device”, the entire disclosure of which is incorporated herein by reference.

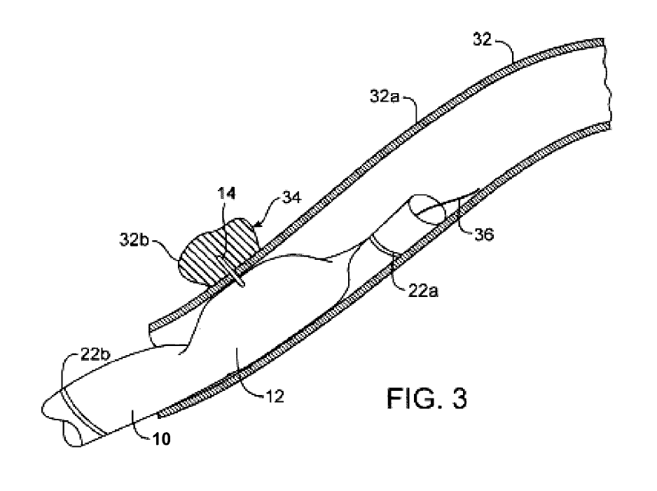

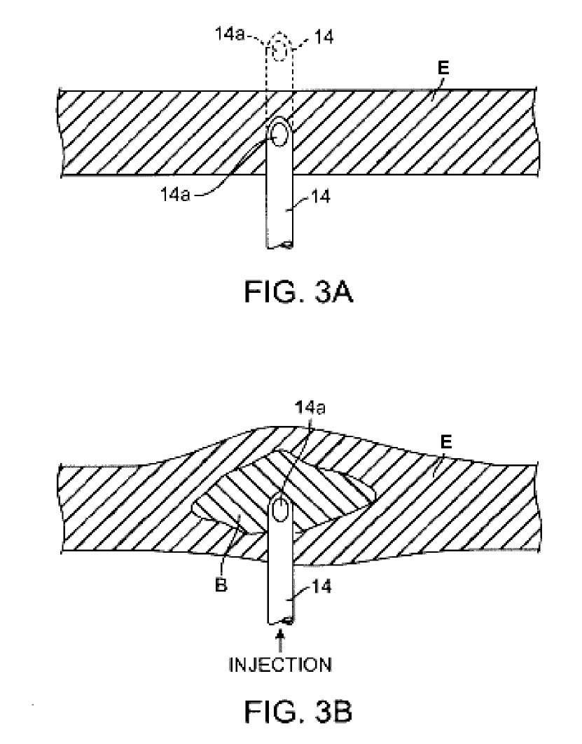

As shown in FIG. 3, the catheter 10, in use, can be inserted through an opening in a patient's body (e.g., for tracheal, bronchial, or sinus access) or through a percutaneous puncture site (e.g. for artery, venous, or tracheal access) and moved within a lumen of the patient 32, until a specific, targeted region 34 is reached (see FIG. 3). The targeted region 34 may be the site of tissue damage, inflammation, or disease, or more usually will be adjacent to these sites typically being within 100 mm or less to allow migration of the pharmaceutical or diagnostic agent. The catheter 20 may follow a guide wire 36 that has previously been inserted into the patient. Optionally, the catheter 10 may also follow the path of a previously-inserted guide catheter, bronchoscope, or tracheoscope (not shown) that encompasses the guide wire. Alternatively or in combination, the catheter may also follow the path of a previously inserted guide catheter, bronchoscope, or tracheoscope without the use of a guide wire.

During maneuvering of the catheter 10, methods of fluoroscopy or magnetic resonance imaging (MRI) can be used to image the catheter and assist in positioning the actuator 12 and the microneedle 14 at the target region. As the catheter is guided inside the patient's body, the microneedle may remain unfurled or held inside the actuator body so that no trauma is caused to the body lumen walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a body lumen wall 32a. It may take only between approximately 100 milliseconds and five seconds for the microneedle to move from its furled state to its unfurled state.

The ends of the actuator at the retaining rings 22a and 22b may remain rigidly fixed to the catheter 10. Thus, they may not deform during actuation. Since the actuator begins as a furled structure, its inflated shape may exist as an unstable buckling mode. This instability, upon actuation, can produce a large-scale motion of the microneedle approximately perpendicular to the central axis of the actuator body, causing a rapid puncture of the body lumen wall without a large momentum transfer. As a result, a microscale opening, or tissue tract, can be produced with very minimal damage to the surrounding tissue. Also, since the momentum transfer can be relatively small, only a negligible bias force may be required to hold the catheter and actuator in place during actuation and puncture.

The microneedle, in fact, can travel with such force that it can enter periluminal tissue 32b, which may include adventitia, media, intima, or any target tissue of interest surrounding body lumens. Additionally, since the actuator is “parked” or stopped prior to actuation, more precise placement and control over penetration of the body lumen wall can be obtained.

Alternatively or in combination, the inflation of the actuator may not result in unstable buckling, but in hydraulic pushing of the needle with the inflation of the balloon. The mechanical advantage offered with the large relative surface area of the balloon pressure focused on the tip of the needle may result in a high force concentration at the needle tip and allow the needle to enter the periluminal tissue 32b.

After actuation of the microneedle and delivery of the pharmaceutical agents to the target region via the aperture of the microneedle, the activating fluid can be exhausted or evacuated from the open area 26 of the actuator, causing the expandable section 24 to return to its original, furled state. This can also cause the microneedle to be withdrawn from the body lumen wall. The microneedle, being withdrawn, can once again sheathed by the actuator.

Various microfabricated devices can be integrated into the needle, actuator and catheter for metering flows, capturing samples of biological tissue, and measuring pH. The catheter 10, for instance, could include electrical sensors for measuring the flow through the microneedle as well as the pH of the pharmaceutical being deployed. The catheter 10 could also include an intravascular ultrasonic sensor (IVUS) for locating vessel walls, and fiber optics, as is well known in the art, for viewing the target region. For such complete systems, high integrity electrical, mechanical and fluid connections may be provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 μm, while the tube's and shaft's exterior cross-sectional dimension may be between about 100 and 500 μm. The overall length of the actuator body may be between about 5 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 50-500 μm. The diameter of the delivery tube for the activating fluid may be about 100 μm to 1000 μm. The catheter size may be between 1.5 and 15 French (Fr). The diameter of the actuator in the actuated, unfurled, or expanded condition may be between 6-16 mm.

Variations of the described embodiments may also utilize a multiple-needle actuator with a single supply tube for the activating fluid. The multiple-needle actuator may include one or more needles that can be inserted into or through a lumen wall for providing injection at different locations or times.

For instance, as shown in FIG. 4, an actuator 120 may include microneedles 140 and 142 located at different points along a length or longitudinal dimension of the central, expandable section 240. The operating pressure of the activating fluid may be selected so that the microneedles move at the same time. Alternatively or in combination, the pressure of the activating fluid may be selected so that the microneedle 140 moves before the microneedle 142.

Specifically, the microneedle 140 may be located at a portion of the expandable section 240 (lower activation pressure) that, for the same activating fluid pressure, may inflate outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 may move before the microneedle 142. It is only when the operating pressure is increased to four psi, for instance, that the microneedle 142 may move. Thus, this mode of operation can provide staged inflation with the microneedle 140 moving at time t₁, and pressure p₁, and the microneedle 142 moving at time t₂ and p₂, with t₁ and p₁ being less than t₂ and p₂, respectively. This sort of staged inflation can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a lumen wall. A needle 224B (as shown in phantom) could alternatively be arranged to move at angle of about 180° to the needle 224A. In general, actuator 220 can be constructed such that one or more needles are arranged with any desirable relative angle to one another.

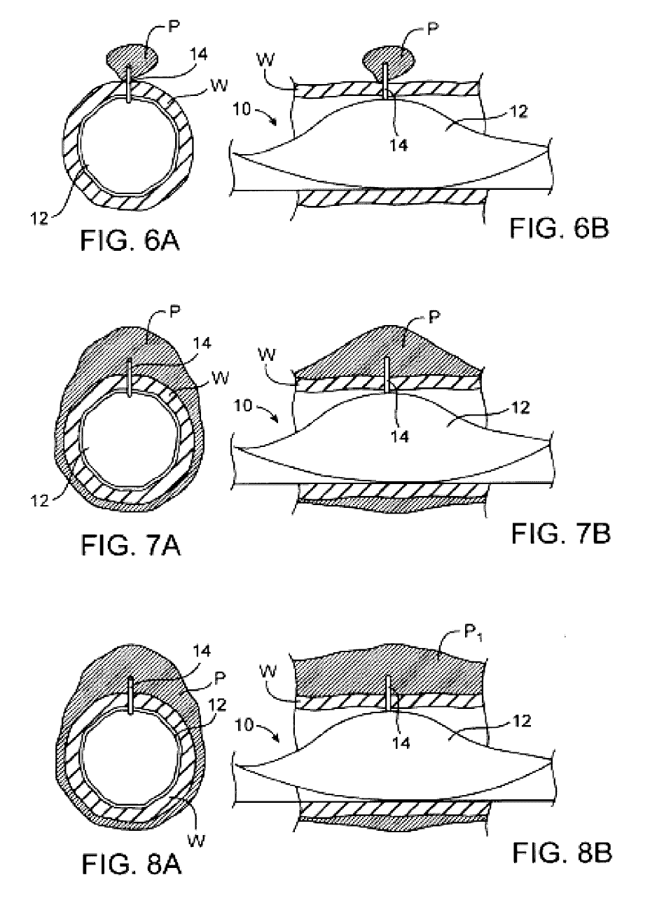

Referring now to FIGS. 6A/6B, 7A/7B, and 8A/8B, use of the catheter 10 of FIGS. 1-5 for delivering a pharmaceutical agent according to the methods of the present invention will be described. The catheter 10 may be positioned so that the actuator 12 is positioned at a target site for injection within a lumen of a patient, as shown in FIGS. 6A/6B. Actuation (e.g. expansion or unfurling) of actuator 12 causes the needle 14 to penetrate through the lumen wall W so that an aperture of needle 14 is positioned into the periluminal space surrounding the lumen wall W. Once in the periluminal space, the pharmaceutical agent, with optionally any contrast or imaging media, may be injected, typically in a volume from 10 μl to 5000 μl, preferably from 100 μl to 1000 μl, and more preferably 250 μl to 500 μl, so that a plume P appears. Initially, the plume occupies a space immediately surrounding the aperture of the needle 14 and extends neither circumferentially nor longitudinally in the periluminal space relative to the exterior of lumen wall W. After a short time, typically in the range from 1 to 10 minutes, the plume extends circumferentially in the periluminal space around the lumen wall W and over a short distance longitudinally, as shown in FIGS. 7A and 7B, respectively. After a still further time, typically in the range from 5 minutes to 24 hours, the plume may extend substantially completely circumferentially, as illustrated in FIG. 8A, and may begin to extend longitudinally over extended lengths, typically being at least about 2 cm, more usually being about 5 cm, and often being 10 cm or longer, as illustrated in FIG. 8B.

Referring now to FIGS. 1D and 1E, a protocol for positioning the aperture 300 of a microneedle 314 for volumetric delivery of a pharmaceutical agent in accordance with the principles of the present disclosure will be described. The microneedle aperture 300 may be positioned from the lumen L using any of the microneedle catheter systems described herein. In particular, aperture 300 of the microneedle 314 may be positioned beyond the pulmonary mucosal epithelium E, as described previously. As shown in this example, aperture 300 of microneedle 314 is positioned in the adventitia A, however aperture 300 of microneedle 314 can be configured for pharmaceutical agent delivery to any target periluminal space of interest. Once in position, the aperture 300 may release the pharmaceutical agent which can then begin to form a plume P, as illustrated in FIG. 1D. By positioning beyond the lumen wall, extensive volumetric distribution of the pharmaceutical agent can be achieved, as shown in FIG. 1E.

Also shown in FIGS. 1D and 1E, a section of exterior surface 24a of expandable section 24 of actuator 12 (which may also be referred to as a section of the expandable element) adjacent the needle 314 may contact the wall of the lumen L when actuator 12 is in its actuated (or unfurled or expanded) state. This contact of exterior surface 24a with the lumen wall around the laterally extended needle 314 can seal the tissue tract of needle 314, thus preventing leakage of pharmaceutical agents delivered from the aperture 300 of needle 314 back into the lumen L.

Because of variability in lumen wall thickness and obstructions which may limit the penetration depth of the needle being deployed, it may often be desirable to confirm that the pharmaceutical agent delivery aperture of the injection needle is present in the target periluminal space of interest. Such confirmation can be achieved in a variety of ways.

Referring to FIGS. 3A, 3B, and 3C, the needle 14 of FIG. 3 can be positioned through a wall of a lumen so that it lies beyond the pulmonary mucosal epithelium E, as shown in the broken line in FIG. 3A. So long as the aperture 14a lies beyond the periphery of the E, successful delivery of a pharmaceutical agent can usually be achieved. To confirm that the aperture 14a lies within in a target periluminal region, a bolus of contrast or imaging media can be injected prior to or simultaneous with delivery of the pharmaceutical agent. If the aperture 14a has not penetrated through the E, as shown in FIG. 3B, then the bolus B of contrast or imaging media may remain constrained within the wall of the lumen forming a well-defined, generally tapered or ovoid mass, as shown in FIG. 3B. In contrast, if the aperture 14a is positioned beyond the E, the bolus B may spread longitudinally in the periluminal space along the lumen wall in a very short period of time, indicating that the drug may be effectively delivered, as shown in FIG. 3C.

As shown in FIG. 3D, other methods for confirming that the aperture 14a is properly positioned may rely on the presence of one or more sensors 15 located on the needle 14 usually near the aperture. One or more sensors 15 may be a solid state pressure sensor. If the pressure builds up during injection (either of an inactive, contrast, or imaging agent or the pharmaceutical agent), the aperture 14a may still lie within the wall of a lumen. If the pressure is lower, the physician may assume that the needle has reached past the lumen wall and into the periluminal space. Sensor 15 may also be a temperature sensor, such as a small thermistor or thermocouple, located at the tip of the needle adjacent to aperture 14a. The temperature within the periluminal space may be different than that at or near the E, making position a function of temperature. The sensor may be a pH detector, where the tissue within the periluminal space versus tissue at or near the E may have detectable differences in pH. Similarly, electrical impedance measurements characteristic of the tissues may be made with an impedance sensor 15. A deflection sensor 17, such as a flexible straining gauge, may be provided on a portion of the needle 14 which can deflect in response to insertion force. Insertion force through the lumen wall may be higher than that necessary to penetrate the tissue beyond the E. Thus, entry into the tissue beyond the E may be confirmed when the insertion force measured by the sensor 17 falls.

The extent of migration of the pharmaceutical agent may not be limited to the immediate periluminal space of the lumen through which the agent is injected. Instead, depending on the amounts injected and other conditions, the pharmaceutical agent may extend further into and through the pulmonary tissue remote from the one or more sites of injection. Delivery and diffusion of a pharmaceutical agent into pulmonary tissue remote from the one or more sites of injection may be useful for treating pulmonary tissue with inflammatory pulmonary disease remote from available body lumen.

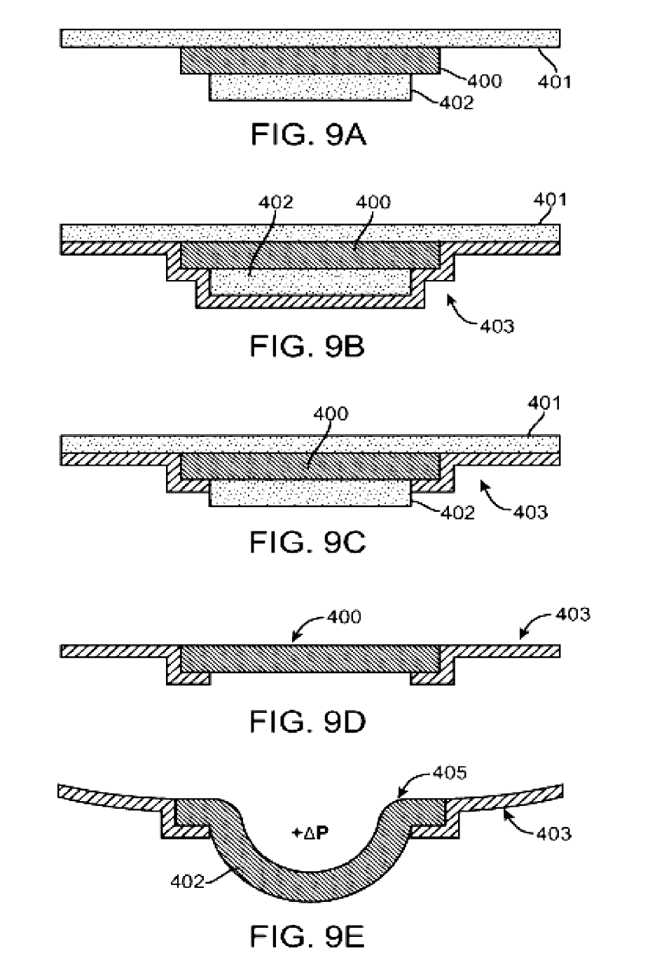

FIGS. 9A, 9B, 9C, 9D, and 9E illustrate an exemplary process for fabricating a dual modulus balloon structure or anchored membrane structure in accordance with the principles of the present disclosure. The first step of the fabrication process is seen in FIG. 9A, in which a low modulus “patch”, or membrane, material 400 is layered between removable (e.g. dissolvable) substrates 401 and 402. The substrate 401 may cover one entire face of the patch 400, while the substrate 402 may cover only a portion of the opposite face, leaving an exposed edge or border region about the periphery.

In FIG. 9B, a layer of a “flexible but relatively non-distensible” material 403 may be deposited onto one side of the sandwich structure from FIG. 9A to provide a frame to which the low-modulus patch is attached. This material may be, for example, Parylene N, C, or D, though it can be one of many other polymers or metals. When the flexible but relatively non-distensible material is Parylene and the patch material is a silicone or siloxane polymer, a chemomechanical bond may be formed between the layers, creating a strong and leak-free joint between the two materials. The joint formed between the two materials usually has a peel strength or interfacial strength of at least 0.05 N/mm², typically at least 0.1 N/mm², and often at least 0.2 N/mm².

In FIG. 9C, the “flexible but relatively non-distensible” frame or anchor material 403 has been trimmed or etched to expose the substrate material 402 so that it can be removed. Materials 401 and 402 may be dissolvable polymers that can be removed by one of many chemical solvents. In FIG. 9D, the materials 401 and 402 may have been removed by dissolution, leaving materials 400 and 403 joined edge-to-edge to form the low modulus, or elastomeric, patch 400 within a frame of generally flexible but relatively non-distensible material 403.

As shown in FIG. 9E, when positive pressure+ΔP is applied to one side 405 of the structure, the non-distensible frame 403 may deform only slightly, while the elastomeric patch 400 may deform much more. The low modulus material may have a material modulus which is always lower than that of the high modulus material and is typically in the range from 0.1 to 1,000 MPa, more typically in the range from 1 to 250 MPa. The high modulus material may have a material modulus in the range from 1 to 50,000 MPa, more typically in the range from 10 to 10,000 MPa. The material thicknesses may range in both cases from approximately 1 micron to several millimeters, depending on the ultimate size of the intended product. For the treatment of most body lumens, the thicknesses of both material layers 402 and 403 are in the range from 10 microns to 2 mm.

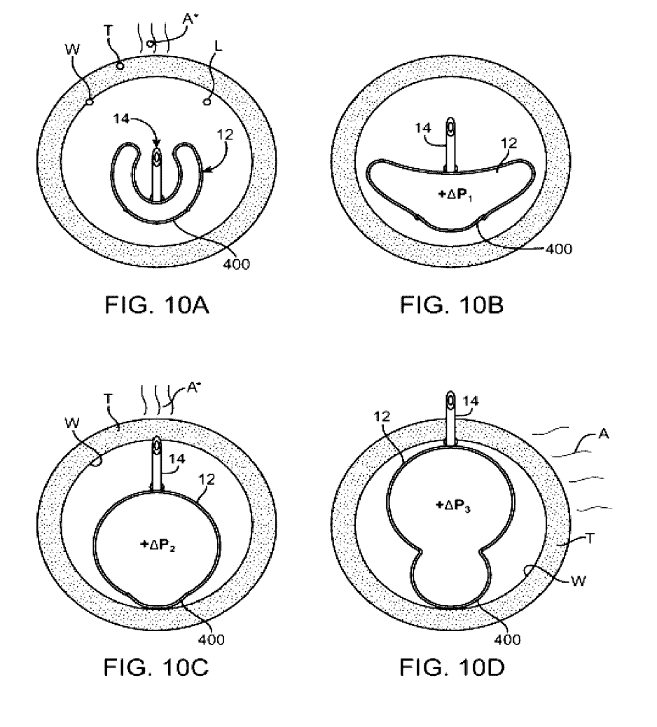

Referring to FIGS. 10A, 10B, 10C and 10D, the elastomeric patch of FIGS. 9A, 9B, 9C, and 9D may be integrated into the intraluminal catheter of FIGS. 1-8. In FIGS. 10A, 10B, 10C and 10D, the progressive pressurization of such a structure is displayed in order of increasing pressure. In FIG. 10A, the balloon may be placed within a body lumen L. The lumen wall W may divide the lumen from periluminal tissue T, or adventitia A*, depending on the anatomy of the particular lumen. The pressure may be neutral, and the non-distensible structure may form a U-shaped involuted balloon 12 similar to that in FIG. 1 in which a needle 14 is sheathed. While a needle is displayed in this diagram, other working elements including cutting blades, laser or fiber optic tips, radiofrequency transmitters, or other structures could be substituted for the needle. For all such structures, however, the elastomeric patch 400 will usually be disposed on the opposite side of the involuted balloon 12 from the needle 14.

Actuation of the balloon 12 may occur with positive pressurization. In FIG. 10B, pressure (+ΔP₁) is added, which can begin to deform the flexible but relatively non-distensible structure, causing the balloon involution to begin its reversal toward the lower energy state of a round pressure vessel. At higher pressure+ΔP₂ in FIG. 10C, the flexible but relatively non-distensible balloon material has reached its rounded shape and the elastomeric patch has begun to stretch. Finally, in FIG. 10D at still higher pressure+ΔP₃, the elastomeric patch has stretched out to accommodate the full lumen diameter, providing an opposing force to the needle tip and sliding the needle through the lumen wall and into the adventitia. Typical dimensions for the body lumens contemplated in this figure may be between 0.1 mm and 50 mm, more often between 0.5 mm and 20 mm, and most often between 1 mm and 10 mm. The thickness of the tissue between the lumen and adventitia may typically be between 0.001 mm and 5 mm, more often between 0.01 mm and 2 mm and most often between 0.05 mm and 1 mm. The pressure+ΔP useful to cause actuation of the balloon may typically be in the range from 0.1 atmospheres to 20 atmospheres, more typically in the range from 0.5 to 20 atmospheres, and often in the range from 1 to 10 atmospheres.

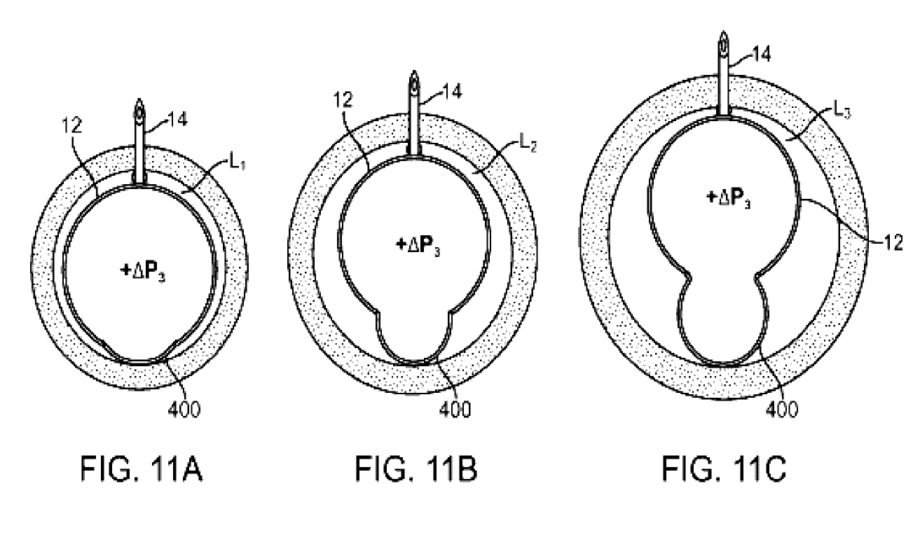

As illustrated in FIGS. 11A, 11B, and 11C, the dual modulus structure formed herein can provide for low-pressure (i.e., below pressures that may damage body tissues) actuation of an intraluminal medical device to place working elements such as needles in contact with or through lumen walls. By inflation of a constant pressure, the elastomeric material may conform to the lumen diameter to provide full apposition. Dual modulus balloon 12 may be inflated to a pressure+ΔP₃ in three different lumen diameters in FIGS. 11A, 11B, and 11C, and the progressively larger inflation of patch 400 provides optimal apposition of the needle through the vessel wall regardless of diameter. Thus, a variable diameter system may be created in which the same catheter may be employed in lumens throughout the body that are within a range of diameters. This can be useful because most medical products are limited to very tight constraints (typically within 0.5 mm) in which lumens they may be used. A system as described in the present disclosure may accommodate several millimeters of variability in the luminal diameters for which they are useful. Further, and as described above, a section of the non-distensible and expandable structure adjacent needle 14 and opposite patch 400 may contact the lumen wall, acting to seal the tissue tract of needle 14 and prevent leakage of pharmaceutical agents delivered from needle 14 back into the lumen.

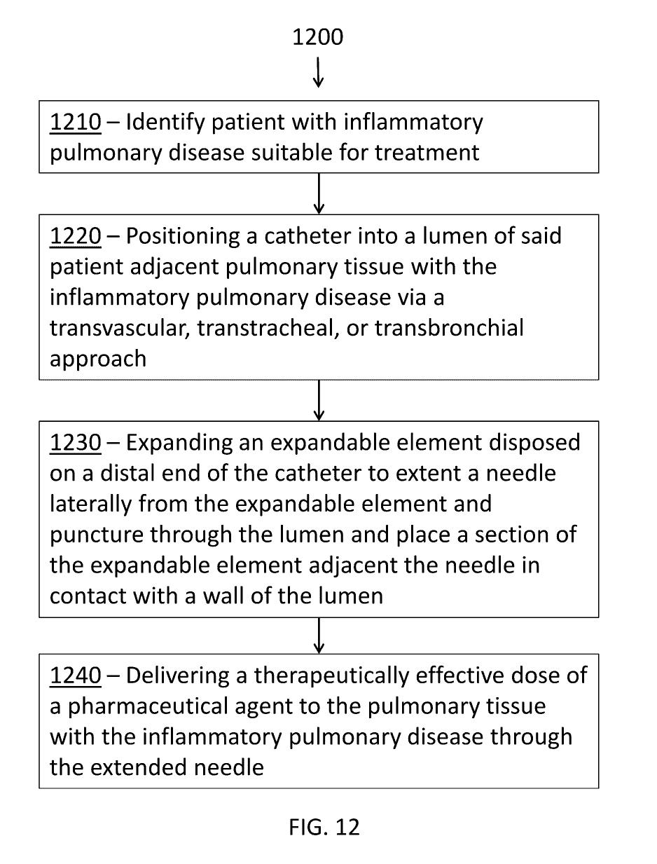

FIG. 12 shows an exemplary method 1200 utilizing the devices and agents described herein for the treatment of a patient with an inflammatory pulmonary disease. Method 1200 may begin with a step 1210 wherein a patient with inflammatory pulmonary disease suitable for treatment may be identified. Once a patient is identified for treatment, one or more suitable pharmaceutical agents may be selected based on the disease, and one or more suitable approaches for delivery of the pharmaceutical agents to one or more target pulmonary tissues may be selected, such as from a transvascular, transtracheal, or transbronchial approach. After determining the suitable delivery approach, a drug delivery catheter as described herein can be positioned into an appropriate lumen of the patient adjacent the target pulmonary tissue with the inflammatory pulmonary disease via the transvascular, transtracheal, or transbronchial approach in a step 1220. In a next step 1230, the actuator, or expandable element disposed on a distal end of the catheter, as described herein, can be expanded to: extend a needle laterally from the expandable element and puncture through the lumen, place the needle in the target periluminal space, and place a section of the expandable element adjacent the needle in contact with the wall of the lumen. A therapeutically effective dose of the pharmaceutical agent can then be delivered to the pulmonary tissue with the inflammatory pulmonary disease through the extended needle, as in a step 1240. While the pharmaceutical agent is being delivered, the section of the expandable element adjacent the needle in contact with a wall of the lumen may seal and prevent leakage of the pharmaceutical agent delivered from the laterally extended needle back into the bodily lumen. Upon completion of treatment, the expandable element may be collapsed and the needle retracted from the lumen wall, allowing for the catheter to be extracted from the lumen of the patient. Potential variations of the aforementioned method will now be described below.

As shown in step 1220, and as previously described, transvascular, transtracheal, or transbronchial administration of a pharmaceutical agent may be performed using the drug delivery catheters herein disclosed. For the transvascular approach, a delivery catheter may be percutaneously advanced through any of a suitable artery or vein or vessel of the patient and placed adjacent the target pulmonary tissue. Exemplary routes to pulmonary tissue may include the advancement of a drug delivery catheter through any of the internal jugular, subclavian, or femoral veins or any of their branches via percutaneous access, further advancing the catheter through the superior or inferior vena cava as appropriate, further advancing the catheter through the right atrium of the heart, further advancing the catheter through the right ventricle of the heart, further advancing the catheter through the pulmonary trunk, then further advancing the catheter through either of the left or right pulmonary arteries, and further advancing the catheter adjacent to a target pulmonary tissue via the pulmonary arteries or downstream vessels. After administration of the pharmaceutical agent is complete, the catheter may be removed.

For the transtracheal approach, a delivery catheter may be advanced into the mouth and then further advanced through the trachea adjacent to a target pulmonary tissue. The delivery catheter may also be advanced further past the trachea and into either of the left or right main bronchus for delivery, or further into any downstream bronchi as necessary to place the catheter adjacent target pulmonary tissue.

Similarly, for the transbronchial approach, a delivery catheter may be advanced through the nose or mouth of a patient and further advanced through the trachea to place the catheter adjacent to a target pulmonary tissue. The delivery catheter may also be advanced further past the trachea and into either of the left or right main bronchus for delivery, or further into any downstream bronchi as necessary to place the catheter adjacent target pulmonary tissue.

In any approach, the use of imaging techniques, including but not limited to MRI, ultrasound, CT, or X-ray, may be used to aid in the placement and advancement of a drug delivery catheter to a position adjacent target pulmonary tissue.

In some embodiments, after expansion of the expandable element of the drug delivery catheter to extend the needle on the expandable element laterally into target pulmonary tissue, contrast or imaging agents may be injected prior to injection of pharmaceutical agents to verify proper placement of the needle. Said contrast or imaging agents may be imaged after injection, and a determination made as to whether the needle of the delivery catheter is in the proper location. The expandable element may be deflated, the needle retracted, and the catheter re-positioned based on the results of imaging after injecting the contrast or imaging agents, and once again deployed after repositioning. This cycle may be repeated as necessary until the needle of the catheter is in the proper position for delivery of the pharmaceutical agent. In others embodiments, the contrast or imaging agent is mixed with the pharmaceutical agent, and all steps above carried out while administering both contrast or imaging agent with pharmaceutical agent.

Although the above steps show the method 1200 of treating a patient in accordance with embodiments, a person of ordinary skill in the art will recognize many variations based on the teaching described herein. The steps may be completed in a different order. The steps may be combined with other described steps of catheter advancement through the anatomy, catheter position verification, drug delivery verification, and the like. Steps may be added or omitted. Some of the steps may comprise sub-steps. Many of the steps may be repeated as often as beneficial to the treatment.

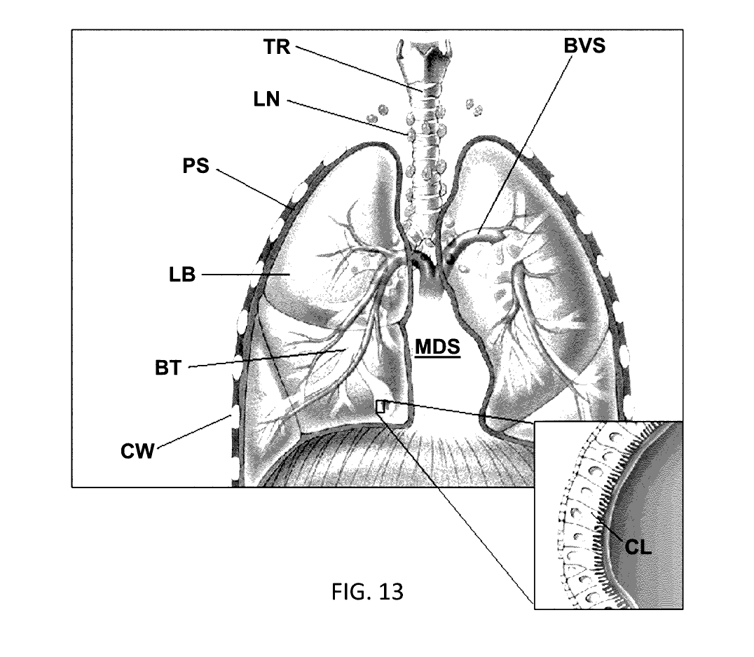

FIG. 13 shows a schematic anterior-view illustration of the gross anatomy of the lung. Starting from the top, the trachea TR may provide pathway for air to enter the lungs and may be a primary pathway of interest for catheter routing. Lymph nodes LN around the trachea as shown are part of the lymph system and may help to prevent illness and infection. Blood vessels BVS are pathways that carry blood into the lungs and throughout the body, and may serve as another pathway of interest for catheter routing. The pleural space PS is the space between the lungs and the chest wall, and is lined on both sides by tissue called pleura. Other anatomical features shown include: the lobes LB of the lung, bronchial tubes BT that serve as air pathways from the trachea to the lungs, the chest wall CW that contains ribs and muscle, the mediastinum MDS which is the space that holds the heart, and cell CL that line internal lumen of tissues of the lung.

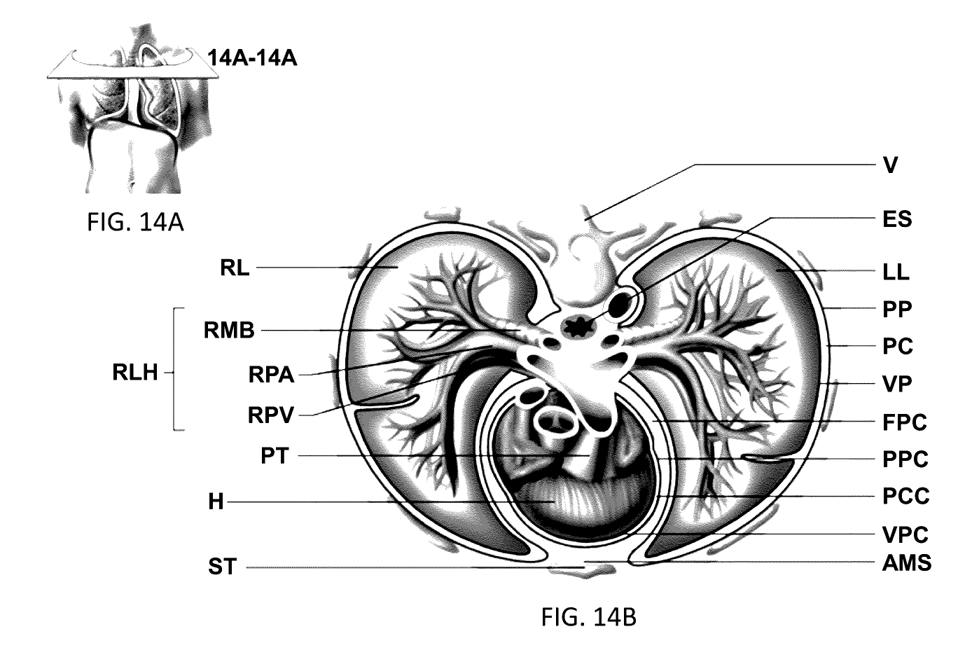

FIG. 14A is a schematic anterior view of a patient defining transverse plane 14A-14A, and accompanying FIG. 14B is a schematic cross-sectional view along transverse plane 14A-14A of FIG. 14A showing the vascularization of the lung. Anatomical features shown include: right lung RL, right main bronchus RMB, right pulmonary artery RPA, right pulmonary vein RPV, pulmonary trunk PT, heart H, sternum ST, root of lung at hilum RLH, vertebra V, esophagus ES, left lung LL, parietal pleura PP, pleural cavity PC, visceral pleura VP, fibrous pericardium FPC, parietal pericardium PPC, pericardial cavity PCC, visceral pericardium VPC, and anterior mediastinum AMS.

### DEFINITIONS

“Pharmaceutical agent” can refer to agents that preferentially inhibit pathogenic molecular or cellular targets or counteract pathophysiologic effects that are identified in a patient with pulmonary inflammatory disease. Examples include, but are not limited to, antibiotic, non-steroidal anti-inflammatory (NSAID), steroid, anti-cytokine antibodies, smooth muscle relaxant, disease modifying anti-rheumatic drug, mucin inhibitor, goblet cell inhibitor, EGFR inhibitor, PDGFR inhibitor, PI3K inhibitor, neurotransmitter receptor inhibitor, protease inhibitor, and the like.

“Antibiotic” can refer to drugs that classically suppress microbial growth, viability or gene expression. Examples are presented in Table 1. It is noted that there are antibiotics with demonstrated efficacy on innate and adaptive immune cell activity, such as metronidazole, azithromycin, erythromycin, clarithromycin and others. It is further noted that in patient populations with chronic inflammatory diseases, disease amelioration may be observed with the administration of antibiotics. This therapeutic effect may be observed in patients that have aberrant Toll-like receptor signaling, uncontrolled tolerance against resident microorganisms in the tissue microbiome, have subclinical persistent infection or have responsive immune cells, or the like.

A list of known toll-like receptors (TLRs) is presented in Table 2.

TABLE 1
Antibiotics as Antagonist Drugs for Pulmonary Inflammatory Diseases
Antibiotics by class

| | | | | |
|---|---|---|---|---|
| Generic name | Brand names | Common uses | Possible side effects | Mechanism of action |

Aminoglycosides

| | | | | |
|---|---|---|---|---|
| Amikacin | Amikin | May be used for | Hearing loss | May bind to the |
| Gentamicin | Garamycin | infections caused | Vertigo | bacterial 30S ribosomal |
| Kanamycin | Kantrex | by Gram-negative | Kidney damage | subunit (some work |
| Neomycin | Neo-Fradin | bacteria, such | | by binding to |
| Netilmicin | Netromycin | as Escherichia | | the 50S subunit), |
| Tobramycin | Nebcin | coli and Klebsiella | | inhibiting the |
| Paromomycin | Humatin | particularly | | translocation of the |
| | | Pseudomonas | | peptidyl-tRNA from |
| | | aeruginosa. | | the A-site to the P- |
| | | May be effective | | site and also causing |
| | | against Aerobic | | misreading of mRNA, |
| | | bacteria (may not | | potentially leaving |
| | | be for | | the bacterium unable |
| | | obligate/facultative | | to synthesize proteins |
| | | anaerobes) | | vital to its growth. |
| | | and tularemia. | | |
| | | Aminoglicocydes | | |
| | | may be ineffective | | |
| | | when taken orally. | | |
| | | Intravenous, | | |
| | | intramuscular and | | |
| | | topical are | | |
| | | preferred. | | |
| Streptomycin | | Tuberculosis | | |
| Spectinomycin(Bs) | Trobicin | Gonorrhea | | |

Ansamycins

| | | |
|---|---|---|
| Geldanamycin | | Experimental, |
| Herbimycin | | as antitumor |
| | | antibiotics |
| Rifaximin | Xifaxan | Traveler's |
| | | diarrhea caused |
| | | by E. coli |

Carbacephem

| | | | |
|---|---|---|---|
| Loracarbef | Lorabid | Discontinued | May prevent bacterial |
| | | | cell division by |
| | | | inhibiting cell wall |
| | | | synthesis. |

Carbapenems

| | | | | |
|---|---|---|---|---|
| Ertapenem | Invanz | Bactericidal for | Gastrointestinal | May inhibit cell wall |
| Doripenem | Doribax | both Gram-positive | upset and | synthesis |
| Imipenem/Cilastatin | Primaxin | and Gram-negative | diarrhea | |
| Meropenem | Merrem | organisms and | Nausea | |
| | | therefore | Seizures | |
| | | potentially useful | Headache | |
| | | for empiric broad- | Rash and allergic | |
| | | spectrum | reactions | |
| | | antibacterial | | |
| | | coverage. (Note | | |
| | | MRSA resistance to | | |
| | | this class.) | | |

Cephalosporins (First generation)

| | | | | |
|---|---|---|---|---|
| Cefadroxil | Duricef | Good coverage | Gastrointestinal | Same mode of action |
| Cefazolin | Ancef | against Gram- | upset and | as other beta-lactam |
| Cefalotin or Cefalothin | Keflin | positive infections. | diarrhea | antibiotics: may |
| Cefalexin | Keflex | | Nausea (if alcohol | disrupt the synthesis |
| | | | taken | of the peptidoglycan |
| | | | concurrently) | layer of bacterial cell |
| | | | Allergic reactions | walls. |

Cephalosporins (Second generation)

| | | | | |
|---|---|---|---|---|
| Cefaclor | Distaclor | Less Gram-positive | Gastrointestinal | Same mode of action |
| Cefamandole | Mandol | coverage, improved | upset and | as other beta-lactam |
| Cefoxitin | Mefoxin | Gram-negative | diarrhea | antibiotics: may |
| Cefprozil | Cefzil | coverage. | Nausea (if alcohol | disrupt the synthesis |
| Cefuroxime | Ceftin, Zinnat | | taken | of the peptidoglycan |
| | (UK) | | concurrently) | layer of bacterial cell |
| | | | Allergic reactions | walls. |

Cephalosporins (Third generation)

| | | | | |
|---|---|---|---|---|
| Cefixime | Suprax | Improved coverage | Gastrointestinal | Same mode of action |
| (antagonistic with | | of Gram-negative | upset and | as other beta-lactam |
| Chloramphenicol) | | organisms, | diarrhea | antibiotics: may |
| Cefdinir | Omnicef, | except potentially | Nausea (if alcohol | disrupt the synthesis |
| | Cefdiel | Pseudomonas. | taken | of the peptidoglycan |
| Cefditoren | Spectracef, | Reduced Gram- | concurrently) | layer of bacterial cell |
| | Meiact | positive coverage. | Allergic reactions | walls. |
| | | May not | | |
| Cefoperazone | Cefobid | cover Mycoplasma | | |
| [Unlike most third- | | and Chlamydia | | |
| generation agents, | | | | |
| cefoperazone may | | | | |
| be active against | | | | |
| Pseudomonas | | | | |
| aeruginosa], | | | | |
| combination | | | | |
| Cefoperazone with | | | | |
| Sulbactam may | | | | |
| make for more | | | | |
| effective antibiotic, | | | | |
| since Sulbactam | | | | |
| may avoid | | | | |
| degeneration of | | | | |
| Cefoperazone | | | | |
| Cefotaxime | Claforan | | | |
| Cefpodoxime | Vantin | | | |
| Ceftazidime | Fortaz | | | |
| [Unlike most third- | | | | |
| generation agents, | | | | |
| ceftazidime is | | | | |
| active | | | | |
| against Pseudomonas | | | | |
| aeruginosa, but | | | | |
| less active against | | | | |
| staphylococci and | | | | |
| streptococci | | | | |
| compare to other | | | | |
| 3rd generation of | | | | |
| Cephalosporins] | | | | |
| Ceftibuten | Cedax | | | |
| Ceftizoxime | Cefizox | | | |
| Ceftriaxone [IV and | Rocephin | | | |
| IM, not orally, | | | | |
| potentially effective | | | | |
| also for syphilis and | | | | |
| uncomplicated | | | | |
| gonorrhea] | | | | |

Cephalosporins (Fourth generation)

| | | | | |
|---|---|---|---|---|
| Cefepime | Maxipime | May covers | Gastrointestinal | Same mode of action |
| | | pseudomonal | upset and | as other beta-lactam |
| | | infections. | diarrhea | antibiotics: may |
| | | | Nausea (if alcohol | disrupt the synthesis |
| | | | taken | of the peptidoglycan |
| | | | concurrently) | layer of bacterial cell |
| | | | Allergic reactions | walls. |

Cephalosporins (Fifth generation)

| | | | | |
|---|---|---|---|---|
| Ceftaroline fosamil | Teflaro | May be used to | Gastrointestinal | Same mode of action |
| | | treat MRSA | upset and | as other beta-lactam |
| | | | diarrhea | antibiotics: may |
| | | | Allergic reaction | disrupt the synthesis |
| | | | | of the peptidoglycan |
| | | | | layer of bacterial cell |
| | | | | walls. |
| Ceftobiprole | Zeftera | May be used to | Gastrointestinal | Same mode of action |
| | | treat MRSA | upset and | as other beta-lactam |
| | | (methicillin-resistant | diarrhea | antibiotics: may |
| | | Staphylococcus | Nausea (if alcohol | disrupt the synthesis |
| | | aureus), penicillin- | taken | of the peptidoglycan |
| | | resistant | concurrently) | layer of bacterial cell |
| | | Streptococcus | Allergic reactions | walls. |
| | | pneumoniae, | | |
| | | Pseudomonas | | |
| | | aeruginosa, and | | |
| | | enterococci | | |

Glycopeptides

| | | | |
|---|---|---|---|
| Teicoplanin | Targocid (UK) | May be active | inhibiting |
| Vancomycin | Vancocin | against aerobic and | peptidoglycan |
| Telavancin | Vibativ | anaerobic Gram- | synthesis |
| Dalbavancin | Dalvance | positive bacteria | |
| Oritavancin | Orbactiv | including MRSA; | |
| | | Vancomycin can be | |
| | | used orally for the | |
| | | treatment of C. | |
| | | difficile | |

Lincosamides(Bs)

| | | | | |
|---|---|---|---|---|
| Clindamycin | Cleocin | Serious staph-, | Possible C. | May bind to 50S |
| Lincomycin | Lincocin | pneumo-, and | difficile-related | subunit of bacterial |
| | | Streptococcal | pseudomembranous | ribosomal RNA, |
| | | infections in | enterocolitis | thereby inhibiting |
| | | penicillin-allergic | | protein synthesis |
| | | patients, also | | |
| | | anaerobic | | |
| | | infections; | | |
| | | clindamycin | | |
| | | topically for acne | | |

Lipopeptide

| | | | |
|---|---|---|---|
| Daptomycin | Cubicin | Gram- | May bind to the |
| | | positive organisms, | membrane and cause |
| | | but may be | rapid depolarization, |
| | | inhibited by | thereby resulting in a |
| | | pulmonary | loss of membrane |
| | | surfactant so less | potential leading to |
| | | effective against | inhibition of protein, |
| | | pneumonias | DNA and RNA |
| | | | synthesis |

Macrolides(Bs)

| | | | | |
|---|---|---|---|---|
| Azithromycin | Zithromax, | Streptococcal | Nausea, vomiting, | May inhibit |
| | Sumamed, | infections, syphilis | and diarrhea | bacterial protein |
| | Xithrone | upper respiratory | (especially at | biosynthesis by |
| Clarithromycin | Biaxin | tract | higher doses) | binding reversibly to |
| Dirithromycin | Dynabac | infections, lower | Prolonged | the subunit 50S of the |
| Erythromycin | Erythocin, | respiratory tract | cardiacQT | bacterial ribosome, |
| | Erythroped | infections | interval | thereby inhibiting |
| Roxithromycin | | mycoplasmal | (especially | translocation of |
| Troleandomycin | Tao | infections, Lyme | erythromycin) | peptidyl tRNA. |
| | | disease | Hearing loss | |
| | | | (especially at | |
| | | | higher doses) | |
| | | | Jaundice | |
| Telithromycin | Ketek | Pneumonia | Visual Disturbance, | |
| | | | Liver Toxicity. | |
| Spiramycin | Rovamycine | Mouth infections | | |

Monobactams

| | | | |
|---|---|---|---|
| Aztreonam | Azactam | Gram-negative | Same mode of action |
| | | bacteria | as other beta-lactam |
| | | | antibiotics: may |
| | | | disrupt the synthesis |
| | | | of the peptidoglycan |
| | | | layer of bacterial cell |
| | | | walls. |

Nitrofurans

| | | |
|---|---|---|
| Furazolidone | Furoxone | Bacterial |
| | | or protozoal |
| | | diarrhea or enteritis |
| Nitrofurantoin(Bs) | Macrodantin, | Urinary tract |
| | Macrobid | infections |

Oxazolidinones(Bs)

| | | | | |
|---|---|---|---|---|
| Linezolid | Zyvox | VRSA | Thrombocytopenia | Protein synthesis |
| | | | Peripheral | inhibitor; may |
| | | | neuropathy | prevent the initiation |
| | | | Serotonin | step |
| | | | Syndrome | |
| Posizolid | Phase II | | | |
| | clinical trials | | | |
| Radezolid | Phase II | | | |
| | clinical trials | | | |
| Torezolid | Phase II | | | |
| | clinical trials | | | |

Penicillins

| | | | | |
|---|---|---|---|---|
| Amoxicillin | Novamox, | Wide range of | Gastrointestinal | Same mode of action |
| | Amoxil | infections; | upset and | as other beta-lactam |
| Ampicillin | Principen | penicillin used | diarrhea | antibiotics: may |
| Azlocillin | | for streptococcal | Allergy with | disrupt the synthesis |
| Carbenicillin | Geocillin | infections, syphilis, | serious | of the peptidoglycan |
| Cloxacillin | Tegopen | and Lyme disease | anaphylactic | layer of bacterial cell |
| Dicloxacillin | Dynapen | | reactions | walls. |
| Flucloxacillin | Floxapen(Sold | | Brain and kidney | |
| | to European | | damage (rare) | |
| | generics | | | |
| | Actavis | | | |
| | Group) | | | |
| Mezlocillin | Mezlin | | | |
| Methicillin | Staphcillin | | | |
| Nafcillin | Unipen | | | |
| Oxacillin | Prostaphlin | | | |
| Penicillin G | Pentids | | | |
| Penicillin V | Veetids (Pen- | | | |
| | Vee-K) | | | |
| Piperacillin | Pipracil | | | |
| Penicillin G | Pfizerpen | | | |
| Temocillin | Negaban (UK) | | | |
| Ticarcillin | Ticar | | | |

Penicillin combinations

| | | | |
|---|---|---|---|
| Amoxicillin/ | Augmentin | Both Amoxicillin/ | The second |
| clavulanate | | clavulanate and | component may |
| | | Ampicillin/ | prevent bacterial |
| | | sulbactam may be | resistance to the first |
| | | effective against | component |
| | | non-recurrent | |
| | | acute Otitis media. | |
| Ampicillin/ | Unasyn | | |
| sulbactam | | | |
| Piperacillin/ | Zosyn | | |
| tazobactam | | | |
| Ticarcillin/ | Timentin | | |
| clavulanate | | | |

Polypeptides

| | | | | |
|---|---|---|---|---|
| Bacitracin | | Eye, ear or bladder | Potential kidney and | May inhibit isoprenyl |
| | | infections; usually | nerve damage (when | pyrophosphate, a |
| | | applied directly to | given by injection) | molecule that carries |
| | | the eye or inhaled | | the building blocks of |
| | | into the lungs; | | the peptidoglycan |
| | | rarely given by | | bacterial cell wall |
| | | injection, although | | outside of the inner |
| | | the use of | | membrane |
| Colistin | Coly-Mycin-S | intravenous colistin | | May interact with the |
| Polymyxin B | | is experiencing a | | Gram- |
| | | resurgence due to | | negative bacterial |
| | | the emergence | | outer |
| | | of multi drug | | membrane and |
| | | resistant organisms. | | cytoplasmic |
| | | | | membrane, displacing |
| | | | | bacterial counter ions, |
| | | | | which destabilizes the |
| | | | | outer membrane. May |
| | | | | act like a detergent |
| | | | | against the |
| | | | | cytoplasmic |
| | | | | membrane, which can |
| | | | | alter its permeability. |
| | | | | Polymyxin B and E |
| | | | | may be bactericidal |
| | | | | even in an isosmotic |
| | | | | solution. |

Quinolones/Fluoroquinolone

| | | | | |
|---|---|---|---|---|
| Ciprofloxacin | Cipro, | Urinary tract | Nausea (rare), | May inhibit the |
| | Ciproxin, | infections, bacterial | irreversible damage | bacterial DNA |
| | Ciprobay | prostatitis, | to central nervous | gyrase or the |
| Enoxacin | Penetrex | community- | system (uncommon), | topoisomerase IV |
| Gatifloxacin | Tequin | acquired | tendinosis (rare) | enzyme, thereby |
| Gemifloxacin | Factive | pneumonia, | | inhibiting DNA |
| Levofloxacin | Levaquin | bacterial diarrhea, | | replication and |
| Lomefloxacin | Maxaquin | mycoplasmal | | transcription. |
| Moxifloxacin | Avelox | infections, | | |
| Nalidixic acid | NegGram | gonorrhea | | |
| Norfloxacin | Noroxin | | | |
| Ofloxacin | Floxin, | | | |
| | Ocuflox | | | |
| Trovafloxacin | Trovan | Withdrawn | | |
| Grepafloxacin | Raxar | Withdrawn | | |
| Sparfloxacin | Zagam | Withdrawn | | |
| Temafloxacin | Omniflox | Withdrawn | | |

Sulfonamides(Bs)

| | | | | |
|---|---|---|---|---|
| Mafenide | Sulfamylon | Urinary tract | Nausea, vomiting, | Folate |
| Sulfacetamide | Sulamyd, | infections (except | and diarrhea | synthesis inhibition. |
| | Bleph-10 | sulfacetamide, used | Allergy (including | May be competitive |
| Sulfadiazine | Micro-Sulfon | for eye infections, | skin rashes) | inhibitors of the |
| Silver sulfadiazine | Silvadene | and mafenide and | Crystals in urine | enzyme |
| Sulfadimethoxine | Di-Methox, | silver sulfadiazine, | Kidney failure | dihydropteroate |
| | Albon | used topically | Decrease in white | synthetase, DHPS. |
| Sulfamethizole | Thiosulfil | for burns) | blood cell count | DHPS may catalyze |
| | Forte | | Sensitivity to | the conversion of |
| Sulfamethoxazole | Gantanol | | sunlight | PABA (para- |
| Sulfanilimide | | | | aminobenzoate) |
| (archaic) | | | | to dihydropteroate, a |
| Sulfasalazine | Azulfidine | | | key step |
| Sulfisoxazole | Gantrisin | | | in folate synthesis. |
| Trimethoprim- | Bactrim, | | | Folate is necessary |
| Sulfamethoxazole | Septra | | | for the cell to |
| (Co-trimoxazole) | | | | synthesize nucleic |
| (TMP-SMX) | | | | acids (nucleic acids |
| | | | | are essential building |
| | | | | blocks |
| | | | | of DNA and RNA), |
| | | | | and in its absence |
| | | | | cells cannot divide. |
| Sulfonamidochrysoidine | Prontosil | | | |
| (archaic) | | | | |

Tetracyclines(Bs)

| | | | | |
|---|---|---|---|---|
| Demeclocycline | Declomycin | Syphilis, | Gastrointestinal | May inhibit the |
| Doxycycline | Vibramycin | chlamydial | upset | binding of aminoacyl- |
| Minocycline | Minocin | infections, Lyme | Sensitivity to | tRNA to the mRNA- |
| Oxytetracycline | Terramycin | disease, | sunlight | ribosome complex. |
| Tetracycline | Sumycin, | mycoplasmal | Potential toxicity | May do so by binding |
| | Achromycin | infections, | to mother and | to the 30S ribosomal |
| | V, Steclin | acnerickettsial | fetus during | subunit in the mRNA |
| | | infections, *malaria | pregnancy | translation complex. |
| | | *Note: Malaria may | Enamel | But Tetracycline may |
| | | be caused by | hypoplasia | not be taken together |
| | | a protist and not a | (staining of | with dairy products, |
| | | bacterium. | teeth; potentially | aluminium, iron and |
| | | | permanent) | zinc minerals. |
| | | | transient | |
| | | | depression of | |
| | | | bone growth | |

Drugs against mycobacteria

| | | | | |
|---|---|---|---|---|
| Clofazimine | Lamprene | Antileprotic | | |
| Dapsone | Avlosulfon | Antileprotic | | |
| Capreomycin | Capastat | Antituberculosis | | |
| Cycloserine | Seromycin | Antituberculosis, | | |
| | | urinary tract | | |
| | | infections | | |
| Ethambutol(Bs) | Myambutol | Antituberculosis | | |
| Ethionamide | Trecator | Antituberculosis | | May inhibits peptide |
| | | | | synthesis |
| Isoniazid | I.N.H. | Antituberculosis | | |
| Pyrazinamide | Aldinamide | Antituberculosis | | |
| Rifampicin | Rifadin, | mostly Gram- | Reddish-orange | Binds to the β subunit |
| (Rifampin in US) | Rimactane | positive and | sweat tears, and | of RNA polymeraseto |
| | | mycobacteria | urine | inhibit transcription |
| Rifabutin | Mycobutin | Mycobacterium | Rash, discolored | |
| | | avium complex | urine, GI symptoms | |
| Rifapentine | Priftin | Antituberculosis | | |
| Streptomycin | | Antituberculosis | Neurotoxicity, | As other |
| | | | ototoxicity | aminoglycosides |

Others

| | | | | |
|---|---|---|---|---|
| Arsphenamine | Salvarsan | Spirochaetal infections | | |
| | | (obsolete) | | |
| Chloramphenicol | Chloromycetin | Meningitis, MRSA, | Rarely: aplastic | May inhibit bacterial |
| (Bs) | | topical use, or for | anemia. | protein synthesis by |
| | | low-cost internal | | binding to the 50S |
| | | treatment. | | subunit of the |
| | | Historic: typhus, | | ribosome |
| | | cholera. Gram- | | |
| | | negative, Gram- | | |
| | | positive, anaerobes | | |
| Fosfomycin | Monurol, | Acute cystitis in | This antibiotic is not | May inactivates |
| | Monuril | women | recommended for | enolpyruvyl |
| | | | children and 75 up | transferase, thereby |
| | | | of age | blocking cell |
| | | | | wall synthesis |
| Fusidic acid | Fucidin | | | |
| Metronidazole | Flagyl | Infections caused | Discolored urine, | May produce |
| | | by anaerobic | headache, metallic | toxic free radicals that |
| | | bacteria; | taste, nausea; alcohol | disrupt DNA and |
| | | also amoebiasis, | is contraindicated | proteins. This non- |
| | | trichomoniasis, | | specific mechanism |
| | | giardiasis | | may be responsible |
| | | | | for its activity against |
| | | | | a variety of bacteria, |
| | | | | amoebae, and |
| | | | | protozoa. |
| Mupirocin | Bactroban | Ointment for | | May inhibit |
| | | impetigo, cream for | | isoleucine t-RNA |
| | | infected cuts | | synthetase (IleRS) |
| | | | | causing inhibition of |
| | | | | protein synthesis |
| Platensimycin | | | | |
| Quinupristin/ | Synercid | | | |
| Dalfopristin | | | | |
| Thiamphenicol | | Gram- | Rash. May lack | A chloramphenicol |
| | | negative, Gram- | known anemic side- | analog. May inhibit |
| | | positive, anaerobes. | effects. | bacterial protein |
| | | Widely used in | | synthesis by binding |
| | | veterinary | | to the 50S subunit of |
| | | medicine. | | the ribosome |
| Tigecycline(Bs) | Tigacyl | Slowly Intravenous. | Teeth discoloration | Similar structure with |
| | | Indicated for | and same side effects | tetracycline, but may |
| | | complicated | as Tetracycline. May | be 5 times stronger, |
| | | skin/skin structure | not be given for | good volume |
| | | infections, soft | children and | distribution and long |
| | | tissues infections | pregnant or lactate | half-time in the body |
| | | and complicated | women. Relatively | |
| | | intra-abdominal | safe and potentially | |
| | | infections. May be | no need dose | |
| | | effective for gram | adjusted when given | |
| | | positive and | for mild tomoderate | |
| | | negative and also | liver function or | |
| | | anaerob antibiotics, | renal patients | |
| | | against multi- | | |
| | | resistant antibiotics | | |
| | | bacterias such as | | |
| | | Staphylococcus | | |
| | | aureus (MRSA) and | | |
| | | Acinetobacter | | |
| | | baumannii, but may | | |
| | | not be effective for | | |
| | | Pseudomonas spp | | |
| | | and Proteus spp | | |
| Tinidazole | Tindamax | Protozoal infections | Upset stomach, bitter | |
| | Fasigyn | | taste, and itchiness | |
| Trimethoprim(Bs) | Proloprim, | Urinary tract | | |
| | Trimpex | infections | | |

NOTE:
(Bs) Bacteriostatic

TABLE 2
TLR ligands and clinically-relevant TLR modulators

| | Exogenous and | TLR Modulators under Clinical Development | |
|---|---|---|---|
| TLR | Endogenous Ligands | Agonists | Antagonists |
| TLR1 | Bacterial lipopeptides | | |
| TLR2 | Bacterial lipoproteins | | OPN-305 (antibody; |
| | and glycolipids | | inflammation, autoimmunity, |
| | Endogenous HMGB1, | | ischemia/reperfusion, |
| | HSP70, EDN, HA, HS | | preclinical) |
| TLR2/TLR1 | Bacterial diacyl | | |
| | lipopeptides | | |
| TLR2/TLR6 | Bacterial triacyl | | |
| | lipopeptides | | |
| TLR3 | Viral double-stranded | AMP-516 (rintatolimod; viral | |
| | RNA | infections, phase II) | |
| | | Poly I: C (vaccine adjuvants, | |
| | | phase III) | |
| TLR4 | Bacterial LPS | Pollinex Quattro (allergy, | NI-0101 (antibody; acute |
| | Endogenous HMGB1, | phase III) | and chronic inflammation, |
| | HSP60, HSP70, EDN, | | preclinical) |
| | HA, HS, Fibrinogen, | | |
| | S100 protein | | |
| TLR5 | Bacterial flagellin | Vax102, flagellin.HuHA, and | |
| | | flagellin.AvHA fusion proteins | |
| | | (vaccine adjuvants: bacterial, | |
| | | viral infections, phase I) | |
| TLR6 | Bacterial triacyl | | |
| | lipopeptides | | |
| | Fungal zymosan | | |
| TLR7 | Viral single-stranded | AZD8848 (asthma and allergic | |
| | RNA | rhinitis, phase IIa) | |
| | | R-848 (resiquimod) (infectious | |
| | | diseases, phase II) | |
| TLR8 | Viral single-stranded | R-848 (resiquimod) (infectious | |
| | RNA | diseases, phase II) | |
| TLR9 | Bacterial and viral CpG- | ISS1018 (adjuvant allergy, phase | |
| | DNA | II) | |
| | | AVE675 (asthma and allergic | |
| | | rhinitis, phase I) | |
| | | IMO-2134 (allergy, asthma, | |
| | | phase I) | |
| | | SAR-21609 (asthma) | |
| TLR10 | Unknown | | |
| TLR11 | Profilin | | |

NOTES:
(EDN): eosinophil-derived neurotoxin;
(HA): hyaluronan;
(HS): heparin sulfate.

“Smooth muscle relaxant (SMR)” may refer to drugs that affect muscle cells to decrease muscle tone. SMRs may be administered to alleviate symptoms such as muscle spasm, pain, hyperresponsiveness, vasoconstriction and others. Examples of SMRs can include: tiotropium bromide, theophylline, hydralazine, clenbuterol, flavoxate, dicycloverine, papaverine, hyoscine hydrobromide, carisoprodol, cyclobenzaprine, metataxalone, methocarbamol, tizanidine, diazepam, baclofen, substance P inhibitors, dantrolene, chlorzoxazone, gabapentin, orphenadrine, or others.

“Steroid” may refer to cyclic organic compounds comprising a four-carbon ring backbone structure, where 3 rings are 6-carbon rings and one 5-carbon ring, with various side chains covalently linked to the steroid backbone structure. The established mechanism of action for steroids may generally be considered to be the induction of gene expression through the activation of cellular steroid receptors, translocation of steroid-bound receptors to the nucleus, recruitment of transfection machinery, and gene expression of a subset of chromosomal genes. Examples of genes upregulated by steroids can include anti-inflammatory cytokines such as TGF-beta, IL10, IL4, IL13 and regulators such as FoxP3, IKB-alpha, SOCS3.

In mammals, treatment can include endogenous, synthetic or natural forms of: steroids such as sex hormones, androgens, estrogens, progestogens, and others; corticosteroids such as glucocorticoids, mineralcorticoids, and others; and anabolic steroids. Examples of glucocorticoid steroids for the treatment of pulmonary inflammatory diseases can include: triamcinolone, cortisone, hydrocortisone, dexamethasone, prednisone, prednisolone, methylprednisolone, betamethasone, budesonide, and others.

“Non-steroidal anti-inflammatory” or “NSAID” may refer to drugs that provide analgesic, antipyretic, or anti-inflammatory effects, wherein their mechanisms of action may be diverse or have yet to be identified. Some of the most characterized mechanisms can include the inhibition of cyclooxygenase-1 and cyclooxygenase-2 inhibitors, prostaglandin and/or thromboxane inhibitors. Prominent NSAIDs can include: aspirin, ibuprofen, naproxen, rofecoxib, celecoxib, diclofenac, indomethacin, ketoprofen, piroxicam, salicylic acid, diflunisal, dexibuprofen, fenoprofen, dexketoprofen, fluriprofen, oxaprozin, loxoprofen, tolmetin, ketorolac, etodolac, sulindac, aceclofenac, nabumetone, meloxicam, tenoxicam, droxicam, lornoxicam, isoxicam, phenylbutazone, mefenamic acid, meclofenamic acid, flufenamic acid, tolfenamic acid, valdecoxib, parecoxib, lumiracoxib, etoricoxib, firocoxib, nimesulide, clonixin, licofelone, and others. Orally administered NSAIDs can increase the risk for irritable bowel disease, gastric bleeding, peptic ulcers, and dyspepsia.

“EGFR inhibitor” may refer to drugs that inhibit the epidermal growth factor receptor, also known as ErbB-1, HER1. EGFR can be a cell-surface receptor located on the surface of many cell types. EGFR ligands can include EGF and transforming growth factor alpha (TGFa). Upon ligand binding, EGFR activation may occur, which can induce the activation of MAPK, Akt, and JNK kinases and can lead to DNA synthesis, cell proliferation, cell migration, or cell adhesion and has been characterized in pulmonary inflammatory disease. Examples of EGFR inhibitors can include: gefitinib, erlotinib, afatinib, brigatinib, icotinib, cetuximab, panitumumab, zalutumumab, nimotuzumab, matuzumab, lapatinib, and others.

“PDGFR inhibitor” may refer to drugs that inhibit the platelet derived growth factor receptor activity. Stimulation of PDGFR leads to angiogenesis, cell growth and cell proliferation. PDGF can be a potent mitogen on fibroblasts and smooth muscle cells in mammals. PDGF can be synthesized and released by numerous cell types including smooth muscle cells, activated myeloid cells such as monocytes, and macrophages and endothelial cells. PDGF binding to PDGFR can lead to the activation of PI3K, STATs, and other signal transducers, and can lead to the regulation of gene expression and a change in cell cycle. Aberrant activity of PDGFR can be characterized in numerous fibrotic diseases, such as pulmonary inflammatory diseases. Examples of PDGFR inhibitors can include: AC 710, AG 18, AP 24534, DMPQ dihydrochloride, PD 166285 dihydrochloride, SU 16f, SU 6668, Sunitinib malate, Toceranib, Gleevec, anti-PDGF neutralizing antibodies, anti-PDGFR antagonist antibodies, and others.

“PI3K inhibitor” or “phosphoinositide 3-kinase inhibitor” may refer to a specific class of drug that can function to inhibit PI3K, which can play a predominant role in the PI3K/AKT/mTOR pathway and can control cellular growth, metabolism, and protein translation. PI3K, which can play a significant role in cell proliferation, can also play a predominant role in cell migration, and the aberrant signaling activity of PI3K in cells can be observed in many fibrotic diseases. Examples of PI3K inhibitors can include: Wortmannin, demethoxyviridin, LY294002, Idelalisib, Perifosine, PX-866, IPI-145, BAY 80-6946, BEZ235, RP6530, TGR 1202, SF1126, INK1117, GDC-0941, BKM120, XL147, XL765, Palomid 529, GSK1059615, ZSTK474, PWT33597, IC87114, TG100-115, CAL263, RP6503, PI-103, GNE-477, CUDC-907, AEZS-136, and others.

“Neurotransmitter receptor inhibitor” may refer to drugs that selectively bind and inhibit the activation or activity of a cellular receptor specific for neurotransmitters. Classes of neurotransmitter receptors that can be present on the surface of activated immune cells, endothelial cells, epithelial cells, and smooth muscle cells involved in pulmonary inflammatory disease can include adrenergic, dopaminergic, GABAergic, glutaminergic, histaminergic, cholinergic, and serotonergic. Examples of neurotransmitter receptor inhibitors can include: propranolol, nadolol, carvedilol, labetalol, oxprenolol, penbutolol, timolol, acebutolol, atenolol, esmolol, metaprolol, nebivolol, sitaxentan, ambrisentan, atrasentan, bosentan, macitentan, tezosentan, chlorpromazine, haloperidol, loxapine, molindone, perphenazine, thioridazine, thiothixene, trifluoperazine, amisulpride, clozapine, olanzapine, quetiapine, risperidone, domperidone, metoclopramide, prochlorperazine, methylphenidate, bupropion, amineptine, ketamine, reserpine, scopolamine, metrazol, diazepam, lorazepam, flumazenil, tizanidine, baclofen, clonazepam, diphenhydramine, doxylamine, chlorpromazine, orphenadrine, quetiapine, cimetidine, famotidine, ciproxifan, thioperamide, and others.

“Protease inhibitor” may refer to drugs that can selectively bind and inhibit the ability of protease enzymes from proteolytically cleaving proteins. Classes of proteases that can inhibit pulmonary inflammatory disease can include: serine proteases, threonine proteases, cysteine proteases, aspartate proteases, glutamic acid proteases, and metalloproteases. Examples can include: members of the serpin family such as alpha-1-antitrypsin, Ci-inhibitor, antithrombin, alpha-1-antichymotrypsin, plasminogen activator inhibitor-1, neuroserpin, and others; antivirals including amprenavir, indinavir, saquinavir, nelfinavir, atazanavir, tipranavir, ritonavir, darunavir, fosamprenavir, lopinavir, ritonavir, telaprevir, cobeprevir, simeprevir, and others; natural inhibitors such as lipocalin proteins; chelators such as EGTA, EDTA, enterochelin, desferroxamine, deferasirox, 1,10-phenanthroline, and others; phosphoramidon; bestatin; alpha-2-macroglobulin; and others.

“DMARDS” may refer to “disease modifying anti-rheumatic drugs”. DMARDs can include an unrelated grouping of drugs traditionally defined by their use in rheumatoid arthritis to retard disease progression in mammals and have since been applied to many other chronic inflammatory diseases that can be autoimmune in nature. Examples of DMARDs for use in pulmonary inflammatory diseases can be found in Table 3.

TABLE 3
DMARDS for use in Pulmonary Inflammatory Diseases.

| Drug | Mechanism |
|---|---|
| abatacept | T-cell costimulatory signal inhibitor |
| adalimumab | TNF inhibitor |
| azathioprine | Purine synthesis inhibitor |
| chloroquine and | Suppression of IL-1 & TNF-alpha, induce |
| hydroxychloroquine | apoptosis of inflammatory cells and |
| (antimalarials) | decrease chemotaxis |
| ciclosporin | calcineurin inhibitor |
| (Cyclosporin A) | |
| D-penicillamine | Reducing numbers of T-lymphocytes etc. |
| (seldom used today) | |
| etanercept | decoy TNF receptor |
| golimumab | TNF inhibitor |
| gold salts (sodium | unknown |
| aurothiomalate, auranofin) | |
| (seldom used today) | |
| infliximab | TNF inhibitor |
| leflunomide | Pyrimidine synthesis inhibitor |
| methotrexate (MTX) | Purine metabolism inhibitor |
| minocycline | 5-LO inhibitor |
| rituximab | chimeric monoclonal antibody against |
| | CD20 on B-cell surface |
| sulfasalazine (SSZ) | Suppression of IL-1 & TNF-alpha, induce |
| | apoptosis of inflammatory cells and |
| | increase chemotactic factors |
| tofacitinib | JAK inhibitor, inhibits innate immune cell |
| | activation and function leading to impaired |
| | T-cell activation |
| ruxolitinib | JAK inhibitor, inhibits dendritic cell |
| | differentiation and function leading to |
| | impaired T-cell activation |

“Anti-cytokine antibody” may refer to any antibody, F(ab) fragment or other variant that can recognize and bind a specific epitope of a cytokine. In this filing the term “antibody” can also mean soluble receptor. Examples of anti-cytokine antibodies can include: infliximab, adalimumab, golimumab, certolizumab, tocilizumab, rituximab, mepolizumab, reslizumab, benralizumab, lebrikizumab, and other antibodies against IL-1, IL-4, IL-5, IL-6, IL-8, IL-13, IL-17, IL-23, IL-33, TNF, or others.

“Activity” of a pharmaceutical agent may refer to, but is not limited to, any enzymatic, allosteric inhibitor, binding function or counter-acting function performed by the agent.

“Comparable cell” may refer to a cell whose type is identical, near identical, or similar to that of another cell to which it is compared. Examples of comparable cells can be cells from the same cell line.

“Inhibiting” or “Antagonizing” may include suppressing or preventing initiation, progression, or relapses of disease, including the progression of established disease. In some embodiments, inhibiting the onset of a disorder means preventing its onset entirely. As used herein, onset may refer to a relapse in a patient that has ongoing relapsing remitting disease. The methods of the invention may be specifically applied to patients that have been diagnosed with an inflammatory disease of the lung or pulmonary tissue. Treatment may be aimed at the treatment or prevention of relapses, which can be an exacerbation of a pre-existing condition. Treatment may also prevent progression of disease symptoms, or may reduce pre-existing symptoms.

“Subject”, “patient”, or “host” may refer to any animal, such as a human, non-human primate, mouse, rat, guinea pig, pig, sheep, cow, rabbit, or others.

“Suitable conditions” may have a meaning dependent on the context in which this term is used. That is, when used in connection with a pharmaceutical agent, the term may refer to conditions that may permit a pharmaceutical agent to bind to its corresponding molecular or cellular target. When used in connection with a pharmaceutical agent that is proteinaceous in nature, the term may refer to conditions that may permit binding of one or more epitopes on said pharmaceutical agent to one or more cognate molecular or cellular targets. When used in connection with contacting an antagonist pharmaceutical agent to a cell, this term may refer to conditions that may permit an agent capable of doing so to bind to a membrane-bound molecular target or to enter a cell and perform its intended function. In some embodiments, the term “suitable conditions” as used herein refers to physiological conditions.

The term “inflammatory” or “inflammation” may refer to: the development processes involving the secretion of cytokines, chemokines, and antibodies; bronchial spasm; hyper-secretion or aberrant accumulation of mucus; hyperproliferation of cells in a tissue; secretion of proteases; tissue remodeling; a humoral (antibody mediated) and/or a cellular (mediated by innate immune cells or antigen-specific T cells or their secretion products) response; or the like. An “immunogen” may be capable of inducing an immunological response against itself upon administration to a mammal or due to an inflammatory pulmonary disease.

The term “transvascular” may refer to across a vessel (artery or vein) wall, from the inside of the vessel to the outside. For example, transvascular drug delivery may describe the delivery of a drug from a source or conduit inside the vessel to the outside of the vessel, such as through a microneedle placed through the vessel wall.

The term “transtracheal” may refer to across the tracheal wall, from the inside of the trachea to the outside of the trachea or to the periluminal tissue within the trachea.

The term “transbronchial” may refer to across the bronchial wall, from the inside of the bronchus, bronchi, or brionchioles to the outside of the bronchus, bronchi, or brionchioles or to the periluminal tissue within the bronchus, bronchi, or brionchioles.

The subject methods may be used for prophylactic or therapeutic purposes. As used herein, the term “treating” may refer to prevention of relapses and/or treatment of pre-existing conditions. For example, the prevention of autoimmune disease may be accomplished by administration of a pharmaceutical agent prior to development of a relapse. The treatment of ongoing disease, where the treatment may stabilize and/or improve the clinical symptoms of a patient, is of particular interest.

Inflammatory diseases of interest may include autoimmune and inflammatory conditions in patients presenting with symptoms consistent to asthma, COPD, pulmonary infection, or the like, wherein disease severity may be characterized as having aberrant inflammatory activity affecting tissues of the lung and related to lung function. Methods of the present disclosure may include administering to a patient an effective amount of a pharmaceutical agent in a manner that can bypass the pulmonary mucosal epithelium to suppress, inhibit, or prevent initiation, progression, or relapses of disease mediated by aberrant inflammation.

Embodiments of the methods described herein may further include treating diseases associated with aberrant activity or activation of myeloid-lineage cells, such as but not limited to dendritic cells, neutrophils, mast cells, eosinophils, monocytes, macrophages, and the like. Myeloid-lineage cell dysfunction may be a major contributor to tissue damage, tissue remodeling and disseminated inflammation in pulmonary disease.

Embodiments of the methods descried herein may further include treating disease associated with pathogenic activity or immune activation mediated by resident microorganisms, such as but not limited to: Acinetobacter spp., Bacillus spp., Burkholderia spp., Clostridium spp., Klebsiella spp., Pseudomonas spp., Serratia spp., Campylobacter spp., Enterococcus spp., Proteus spp., Staphylococcus spp., Streptococcus spp., Legionella spp., Mycobacterium spp., Mycoplasma spp., Neisseria spp., Aspergillus spp., Cryptococcus spp., Candida spp., Pneumocystis spp., Histoplasma spp., Sporotrichus spp., Blastomyces spp., and others. In some cases, the present disclosure may provide methods for treating a patient that smoke cigarettes, uses breathing apparati such as an oxygen tank, intubation, or other, or who may be occupationally exposed to high burdens of pulmonary pathogens.

Embodiments of the methods descried herein may further include treating disease associated with aberrant activity of smooth muscle cells, which can account for the hypercontractility, bronchial inflammation, and tissue remodeling observed in inflammatory pulmonary disease. Hypercontraction of smooth muscle cells may involve aberrantly high concentrations of pro-contractile mediators and/or a low concentration of relaxant mediators. Smooth muscle cells can display pro-inflammatory and immunomodulatory functions through the secretion of soluble effectors. In response to inflammatory mediators, smooth muscle cells can also undergo a proliferative response and may be observed in some patients with inflammatory pulmonary conditions such as asthma and COPD.

Embodiments of the methods descried herein may further provide information on growth factor receptor antagonism as it relates to pulmonary inflammatory disease. Members of the growth factor receptors conserved transmembrane receptor family may be constitutively expressed or induced on the surface of most cell types including, but not limited to, immune cells, endothelial, epithelial, stromal cells, and the like. Members can include platelet derived growth factor receptor (PDGFR), epithelial growth factor receptor (EGFR), fibroblast growth factor (FGFR), or the like. Activation of growth factor receptors may lead to: downstream kinase activity; transcription factor activity; and cellular responses such as proliferation, cytokine, and chemokine secretion, cell adhesion molecule expression, metalloproteinase secretion and anti-apoptotic effector functions.

### Conditions for Analysis and Therapy

The compositions and methods of the present disclosure may find use in combination with a variety of inflammatory pulmonary conditions, which include, without limiting, the following conditions.

Asthma.

Asthma can be a complex disease and can display disease heterogeneity and variability in its clinical expression (see Table 4 below). Heterogeneity can be influenced by factors including age, sex, socioeconomic status, ethnicity, genetics and environment. Diagnosis of asthma can often be based on symptoms, for example, airway airflow obstruction, airway inflammation and hyper-responsiveness, and response to therapy over time. Although current treatment modalities may be capable of controlling symptoms and some may improve pulmonary function in some patients, acute and severe exacerbations may still occur, contributing to significant morbidity and mortality in all age groups.

Factors that can increase asthma risk include viral respiratory tract infections in infancy, occupational exposures in adults, and allergen exposure in sensitized individuals. In patients with established asthma diagnoses, disease exacerbations can vary among and within patients, and can include allergen exposure, viral infections, exercise, exposure to irritants, ingestions of nonsteroidal anti-inflammatory agents, and others. Additionally, asthma can be linked to hypervascularity and high levels of angiogenic factors present in tissue biopsies, which may indicate a role of inflammation and angiogenesis or lymphangiogenesis.

Treatment of asthma can be determined largely by the initial clinical assessment of disease severity and the establishment of control of disease symptoms following intervention. Disease severity and control can vary over time for an individual patient. Treatment selection may be evaluated based on current impairment and long-term risk of persistent therapy. Unfortunately, despite the availability of effective therapies, many patients world-wide demonstrate suboptimal asthma control.

Chronic Obstructive Pulmonary Disease, COPD.

Chronic obstructive pulmonary disease (COPD) can be a complex disorder with several unique age-related aspects (see Table 4 below). Underlying changes in pulmonary lung function and poor sensitivity to bronchoconstriction and hypoxia with advancing age can place older adults at greater risk of mortality or other complications from COPD. COPD can be characterized and defined by limitation of expiratory airflow. This can result from several types of anatomical lesions, including loss of lung elastic recoil, fibrosis, and narrowing of small airways. Inflammation, edema, and secretions can also contribute variably to airflow limitation.

Smoking can cause COPD through several mechanisms. First, smoke can be a powerful inducer of an inflammatory response. Inflammatory mediators, including oxidants and proteases, are believed to play a major role in causing lung damage. Smoke can also alter lung repair responses in several ways. Inhibition of repair may lead to tissue destruction that characterizes emphysema, whereas abnormal repair can lead to the peri-bronchiolar fibrosis that can cause airflow limitation in small airways. Genetic factors can likely play a major role and may account for much of the heterogeneity susceptibility to smoke and other factors. Many factors may play a role, but to date, alpha-1 protease inhibitor deficiency has been unambiguously identified. Exposures other than cigarette smoke can contribute to the development of COPD. Inflammation of the lower respiratory tract that can result from asthma or other chronic disorders may also contribute to the development of fixed airway obstruction. COPD may not only be a disease of the lungs but may also be a systemic inflammatory disorder. Muscular weakness, increased risk for atherosclerotic vascular disease, depression, osteoporosis, and abnormalities in fluids and electrolyte balance may all be consequences of COPD.

Effective diagnostic criteria for COPD has been developed by the Global Initiative for Obstructive Lung Disease criteria, which can be effectively applied to patients suspected of having COPD to more rigorously define the diagnosis and management of COPD. An important component of this approach is the use of spirometry for disease staging, a procedure that can be performed in most patients. The management of COPD can includes smoking cessation, influenza and pneumococcal vaccinations, the use of short- and long-acting bronchodilators, and the like.

Unlike with asthma, corticosteroid inhalers can represent a third-line option for COPD. Combination therapy may frequently be required. When using various inhaler designs, it is important to note that older adults, especially those with more-severe disease, may have inadequate inspiratory force for some dry-powder inhalers, although many older adults may find the dry-powder inhalers easier to use than metered-dose inhalers. Other treatments include pulmonary rehabilitation, oxygen therapy, noninvasive positive airway pressure, depression and osteopenia screening, and the like.

TABLE 4
Airway disease classification.

| | | | |
|---|---|---|---|
| Disease | Post- | FEV1 | |
| and | bronchodilator | Predicted | |
| Severity | FEV1/FVC | (%) | Symptoms |

COPD

| | | | |
|---|---|---|---|
| Stage I: | <0.7 | ≧80 | Chronic cough, sputum |
| mild | | | production may be present |
| Stage II: | <0.7 | 50-80 | Shortness of breath, cough, |
| moderate | | | and sputum production |
| Stage III: | <0.7 | 30-50 | Greater shortness of breath, |
| severe | | | reduced exercise capacity, |
| | | | fatigue, repeated exacerbations |
| Stage IV: | <0.7 | <30 | Chronic respiratory failure |
| very severe | | | (PaO2 < 8 kPa, PaCO₂ > 6.7 |
| | | | kPa at sea level) |

Asthma

| | | | |
|---|---|---|---|
| Intermittent | ≧0.7 | ≧80 | Shortness of breath, chest |
| | | | tightness, tachycardia, |
| | | | wheezing less than once a |
| | | | week |
| Mild | ≧0.7 | ≧80 | Symptoms intermittent less |
| | | | than once a week, more than |
| | | | once per day |
| Moderate | ≧0.7 | 60-80 | Symptoms intermittent daily |
| persistent | | | |
| Severe | ≧0.7 | <60 | Symptoms intermittent daily |
| persistent | | | associated with night-time |
| | | | symptoms |

Pulmonary Infections.

Pulmonary infections may arise from the interaction of lung tissue with pulmonary microorganisms. Persistent infection (from refractory systemic treatment or from antibiotic-resistant microbes) can result in acute bronchitis or pneumonia, and severe infection can result in pulmonary edema, respiratory failure, and death. Pulmonary infections are often caused by viruses, but also can be caused by bacteria or fungal organisms. Microorganisms responsible may enter the lung by the following routes: via the tracheobronchial tree, most commonly due to smoking or the inhalation of droplets of secretions from another infected human or also environmental exposure (e.g. fungal spores); via the pulmonary vasculature, usually due to direct injection (e.g. intravenous drug use) or secondary seeding from distant infection (e.g. infective bacterial endocarditis); or via direct spread from infection in the mediastinum, chest wall, or upper abdomen.

With rest, supportive care, and the administration of antibiotics and anti-inflammatory agents, most of these infections can improve in a few weeks, however a subset of patients do not improve upon oral or intravenous antibiotic administration. In these patients, symptoms can persist beyond a few weeks and may indicate a more complicated infection and result in more significant tissue morbidity. Symptoms suggestive of a chronic and/or resistant infection can include: fevers for over 1 week, cough for over 3 weeks, swollen lymph nodes (glands) in the neck or arm pits, coughing up blood, or feeling like symptoms return after cessation of antibiotic therapy. Often, patient evaluation may include repeated x-rays, CAT scans, bacterial or fungal growth assays, and occasionally bronchoscopy. Many chronic pulmonary infections can be treatable, especially when diagnosed early. Specific infections related to the present disclosure include, but are not limited to: 1) Histoplasmosis, a fungus that can live in the soil and can be associated with bird droppings. Histoplasmosis may not be passed person to person. It can cause either an acute or chronic pneumonia. It can be treatable and is often diagnosed by a blood or urine test. 2) Blastomycosis, a fungus that lives in the soil. Like Histoplasmosis it is may not be passed person to person. It can cause chronic pneumonias and skin sores that resemble boils. It can often be diagnosed by blood test or examination of the sores on the skin. It can be treatable with antifungal agents. 3) Tuberculosis (TB), caused by Mycobacterium tuberculosis bacteria can cause chronic pneumonia. It is highly contagious and can be passed person to person by aerosolized infectious units. Often those exposed may not develop pneumonia immediately, and may require treatment with liver-toxic antibiotics such as isoniadiz prior to development of a true infection. 4) Mycobacterium (non-tuberculosis). These close relatives may not be passed person to person and may generally be acquired from the soil or water contamination. These bacteria can cause a chronic infection that may need to be treated. 5) Bronchiectasis, which may not be a true infection. Patients with bronchiectasis may have scarring in their lungs which can make them susceptible to repeat bouts of bronchitis and pneumonia. There are treatments that can greatly reduce the frequency of these infections. Often bronchiectasis can be diagnosed by a CAT scan of the chest.

In addition to environmental pulmonary infectious agents, community-acquired pneumonias can result from pulmonary infection by: Mycoplasma pneumoniae, Chlamydia pneumoniae, Haemonphilus influenzae, Legionella pneumophila, Moraxella catarrhalis, Staphylococcus aureus, Actinobacillus gordonii, Actinobacillus pleuropneumonias, Actinomyces spp., Streptococcus spp., Pseudomonas spp., Acinetobacter spp., and others.

Additionally, hospital-acquired pneumonias (HAPs) may arise from hospital-related exposures of inhaled microbial load through contaminated ventilators, air and/or aspiration. These HAPs can derive from common and drug-resistant microbes, including but not limited to: Aspergillus spp., Candida spp., Mucor spp., Histoplasma spp., Coccidiodes spp., Blastomyces spp., Paracoccidioides spp., Sorothrix spp., Cryptococcus spp., Mycoplasma pneumoniae, Chlamydia pneumoniae, Haemonphilus influenzae, Legionella pneumophila, Moraxella catarrhalis, Staphylococcus aureus, Actinobacillus spp., Actinomyces spp., Streptococcus spp., Pseudomonas spp., Acinetobacter spp., and others.

A patient with a pulmonary infection may be diagnosed by methods including positive chest x-ray, CT, polymerase chain reaction test, next generation sequencing results, immunoassay results or positive microbial growth culture from sputum, lung lavage or pulmonary aspirate samples. Selection of appropriate one or more pharmaceutical agents can be determined, if necessary, to select for narrow- or broad-spectrum targets.

Embodiments of the methods described herein may further provide for single administration of pharmaceutical agents to the pulmonary tissue by transvascular, transtracheal, or transbronchial injection for the treatment of asthma, COPD or pulmonary infection. Transvascular, transtracheal, or transbronchial injection can be administered in a single dose or periodically as needed to prevent, control or treat established pulmonary inflammatory diseases.

Embodiments of the methods described herein may also provide for combination therapy, where the combination may provide for additive or synergistic benefits. One or more pharmaceutical agents may be combined and selected from one or more of the general classes of drugs commonly used in the treatment of pulmonary inflammatory disease. Long-term control drugs can include, but are not limited to: corticosteroids; cromolyn sodium and nedocromil; immunomodulators such as omalizumab (anti-IgE); leukotriene modifiers such as montelukast, safirlukast; 5-lipoxygenase inhibitors such as zileuton; LABAs such as the bronchodilators salmeterol and formoterol; methylxanthines such as theophylline; and others. Quick-relief drugs can include, but are not limited to: anticholinergics such as ipratropium bromide; SABAs including albuterol, levalbuterol and pirbuterol; systemic corticosteroids; and others.

Other pharmaceutical agents for use in combination with pharmaceutical agents described herein may include non-antigen specific agents used in the treatment of autoimmune disease, which can include corticosteroids and disease modifying drugs, or may include antigen-specific agents. These agents can include: methotrexate, leflunomide (Arava™), etanercept (Enbrel™), infliximab (Remicade™), adalimumab (Humira™), anakinra (Kineret™), rituximab (Rituxan™), CTLA4-Ig (Abatacept™), toclizumab (Actemra™), sarilumab, olokizumab, elsilimomab, CNTO 328, ALD518/BMS-945429, CNTO 136, CPSI-2364, CDP6039, Ruxolitinib, Tofacitinib, Baricitinib, CYT387, Filgotinib, GSK2586184, lestaurtinib, pacritinib, TG101348, antimalarials, sulfasalazine, d-penicillamine, cyclosporin A, cyclophosphamide, azathioprine, and the like. Of particular interest are combinations with drugs targeting TNF, including but not limited to etanercept (Enbrel™), infliximab (Remicade™), and adalimumab (Humira™). Combination of such drugs with pharmaceutical agents may allow for a more sparing use of the pharmaceutical agents.

Anticholinergic, antimuscarinic, or antiocholinergic drugs, for example, scopolamine, clonidine, atropine, diphenhydramine, tiotropium, and the like, can be used to block the activity of neurotransmitter receptors located on the surface of activated immune cells, smooth muscle cells, pulmonary fibroblasts, and epithelial cells. These aforementioned cells may directly mediate disease activity and progression in patients with pulmonary inflammatory diseases. Preferred may be the use of neurotransmitter receptor antagonists to treat lymph nodes, related lymphatics, and surrounding tissues in the lung of patients with pulmonary inflammatory diseases. Also preferred may be the use of neurotransmitter receptor antagonists to treat smooth muscle and tissue surrounding the trachea, bronchus, and bronchi of the lung in patients with pulmonary inflammatory disease. Additionally, said neurotransmitter receptor antagonist drugs may further be used to affect nerves to achieve a more subtle disease modifying affect a patient with COPD, asthma, and pulmonary inflammatory disease activity and progression.

Corticosteroids, e.g. prednisone, methylpredisone, prednisolone, dexamethasone, triamcinolone, solumedrol, etc. may have both anti-inflammatory and immunoregulatory activity. They can be administered orally but are often administered by aerosolization with an inhaler or nebulizer. Corticosteroids may be useful in early disease as a temporary adjunctive therapy while waiting for the pharmaceutical agents to exert their effects. Corticosteroids may also be useful as chronic adjunctive therapy in patients with severe disease. The broad action of corticosteroids on the inflammatory process may account for their efficacy as preventive therapy. Their clinical effects may include: reduction in severity of symptoms; improvement in asthma control and quality of life; improvement in PEF and spirometry; diminished airway hyperresponsiveness; prevention of exacerbations; reduction in systemic corticosteroid courses, ED care, hospitalizations, and deaths due to asthma; and possibly the attenuation of loss of lung function in adults. The clinical effects of corticosteroids can depend on specific anti-inflammatory actions. Corticosteroids may suppress the generation of cytokines, recruitment of airway eosinophils, and release of inflammatory mediators. These anti-inflammatory actions of corticosteroids have been noted in clinical trials and analyses of airway histology. The anti-inflammatory effects of corticosteroids may be mediated through receptors that modulate inflammatory gene expression.

Disease modifying anti-rheumatoid drugs, or DMARDs, have been shown to alter the disease course and improve radiographic outcomes in rheumatoid arthritis. It will be understood by those of skill in the art that these drugs may also be used in the treatment of other autoimmune diseases.

Methotrexate (MTX) is a frequent first-line agent because of its early onset of action (4-6 weeks), good efficacy, favorable toxicity profile, ease of administration, and low cost. MTX is the only conventional DMARD agent in which the majority of patients continue on therapy after 5 years. MTX may be effective in reducing the signs and symptoms of numerous inflammatory diseases. Although the immunosuppressive and cytotoxic effects of oral MTX may be in part due to the inhibition of dihydrofolate reductase, the anti-inflammatory effects in several chronic inflammatory diseases appear to be related at least in part to interruption of adenosine and TNF pathways. The onset of action or oral MTX administration can be 4 to 6 weeks, with 70% of patients having some response.

### Pharmaceutical Compositions

Pharmaceutical agents described herein may serve as the active ingredient in pharmaceutical compositions formulated for the treatment of various disorders as described herein, and can include the use of currently available medications, excipients, solvents, diluents, and others.

The active ingredient can be present in a therapeutically effective amount, i.e., an amount sufficient when administered to treat a disease or medical condition. The compositions can also include various other agents to enhance delivery and efficacy, e.g. to enhance delivery and stability of the active ingredients.

Thus, for example, the compositions may also include, depending on the formulation desired, pharmaceutically-acceptable, non-toxic carriers such as PEG or diluents, which may be defined as vehicles commonly used to formulate pharmaceutical compositions for animal or human administration. The diluent can be selected so as not to affect the biological activity of the combination. Examples of such diluents include, but are not limited to, distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents, and others. The composition can also include any of a variety of stabilizing agents, such as an antioxidant.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via transvascular, transtracheal and/or transbronchial method.

For administration by injection, the active ingredient can be administered in liquid dosage forms, such as suspensions, solutions, emulsions, or the like. The active component(s) can be mixed with inactive ingredients or excipients such as carrier molecules, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion or other known desirable features can include red iron oxide, silica gel, sodium lauryl sulfate, titanium dioxide, edible white ink, and the like.

The active ingredient, alone or in combination with other suitable components, can be made into injectable formulations (i.e., they can “disseminate into tissue”) from the original injection site. Disseminating formulations can be placed into pressurized acceptable propellants, such as dichlorodifluoromethane, propane, nitrogen, and the like.

Formulations suitable for transvascular, transtracheal or transbronchial administration, such as, for example, by transarterial (via an artery) and transvenous (via a vein) methods, may include aqueous and non-aqueous, isotonic sterile injection solutions, which can contain antioxidants, buffers, bacteriostatics, and solutes that render the formulation isotonic with the target pulmonary tissue of the intended recipient, and aqueous and non-aqueous sterile suspensions that can include suspending agents, solubilizers, thickening agents, stabilizers, preservatives, and the like.

Formulations suitable for transvascular, transtracheal or transbronchial administration may also include carriers or excipients intended to extend pharmacokinetics of the active pharmaceutical agent, such as by long-term elution from a polymeric carrier. Such carriers may include nanoparticles, microparticles, nano- or micro-beads comprised of polymers such as poly-lactic acid or the like, self-assembling polypeptides, silk protein, hydrogels, gels, foams, cyclodextrins, or other solutions that polymerize or precipitate upon contact with physiologic conditions but remain in solution when outside the body.

The components used to formulate the pharmaceutical compositions are preferably of high purity and substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### Methods of Treatment

The periodicity of administrating effective doses of a pharmaceutical agent may be on a daily, weekly, or on a periodic or one-time basis. In some embodiments, the pharmaceutical agent can be administered through a transvascular, transtracheal, or transbronchial route once a day to provide therapeutic effects. In other embodiments, effective doses of pharmaceutical agent may be administered through the aforementioned routes once every three days to provide therapeutic effects. In other embodiments, effective doses of pharmaceutical agent may be administered once every week to provide therapeutic benefit. In other embodiments, effective doses of pharmaceutical agent may be administered once every other week to provide therapeutic benefit. In other embodiments, effective doses of pharmaceutical agent may be administered once a month to provide therapeutic benefit. In other embodiments, effective doses of pharmaceutical agent may be administered periodically or as required to provide therapeutic benefit. In some embodiments, distinct and remote pulmonary sites may be treated during the same procedure.

Determining a therapeutically or prophylactically effective amount of pharmaceutical agent can be done based on animal data using routine computational methods. In some embodiments, the therapeutically or prophylactically effective amount contains between 0.00000001-50 mg/kg patient weight. In another embodiment, the effective amount contains between about 0.000001-2.5 mg/kg patient weight, as applicable. In a further embodiment, the effective amount contains between about 0.0001-0.1 mg/kg patient weight, as applicable. The effective dose will depend at least in part on the route of administration and severity of disease symptoms.

In some embodiments, the pharmaceutical agent may be delivered by a drug delivery system consisting of a hydrogel, gel, foam, solution, or suspension.

The pharmaceutical agent compositions may be administered in a pharmaceutically acceptable excipient. The term “pharmaceutically acceptable” may refer to an excipient acceptable for use in the pharmaceutical and veterinary arts, which is not toxic or otherwise inacceptable. The concentration of pharmaceutical agent in the pharmaceutical formulations can vary widely, i.e. from less than about 0.1%, usually at or at least about 2% to as much as 20% to 50% or more by weight, and can be selected primarily by fluid volumes, viscosities, etc., in accordance with the particular mode of administration selected and desired tissue dissemination from the injection site.

In some embodiments, the pharmaceutical agent can be delivered by transvascular injection to the periluminal tissue adjacent to a vessel of the lung. The lung comprises tissues that are rich in blood and lymph vessels, which can rapidly uptake and disseminate pharmaceutical agents from a single injection site for rapid targeting and interruption of disease-causing targets. These advantages confer a faster onset of action with a lower dose when compared to oral or i.v. administration where pharmaceutical agents must pass through some or all of the digestive or circulatory tract in order for absorption to occur.

For the transvascular approach, a delivery catheter of any of the embodiments disclosed herein may be percutaneously advanced through any of a suitable artery or vein or vessel of the patient and placed adjacent the target pulmonary tissue. Exemplary routes to pulmonary tissue may include the advancement of a drug delivery catheter through any of the internal jugular, subclavian, or femoral veins or any of their branches via percutaneous access, further advancing the catheter through the superior or inferior vena cava as appropriate, further advancing the catheter through the right atrium of the heart, further advancing the catheter through the right ventricle of the heart, further advancing the catheter through the pulmonary trunk, then further advancing the catheter through either of the left or right pulmonary arteries, and further advancing the catheter adjacent to a target pulmonary tissue via the pulmonary arteries or downstream vessels. After administration of the pharmaceutical agent is complete, the catheter may be removed.

In some embodiments, the pharmaceutical agent can be delivered by transtracheal or transbronchial injection. Absorption of pharmaceutical agents by cells in the periluminal tissue adjacent to the trachea may bypasses degradation or neutralization in the gastrointestinal tract or in other routes. The number of FDA-approved polymers for use as transdermal delivery agents is increasing rapidly and can be re-purposed for transtracheal injection.

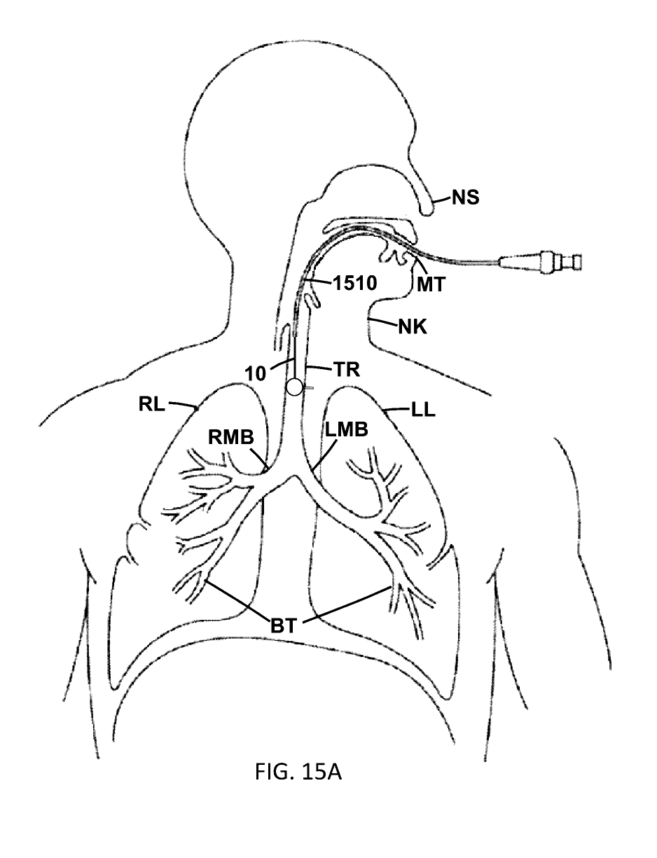

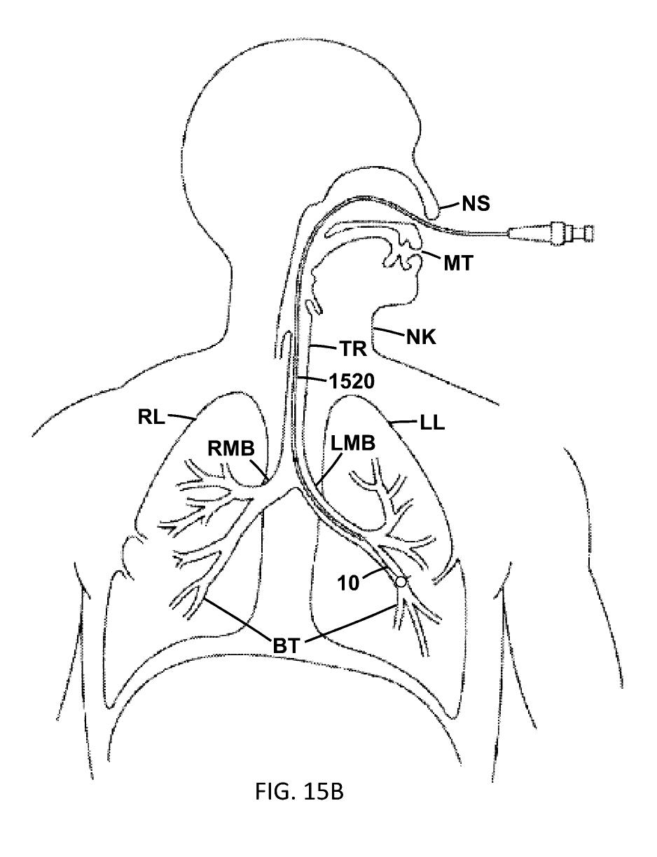

As shown in FIG. 15A, for transtracheal administration, a delivery catheter of any of the embodiments disclosed herein may be advanced through the mouth MT (or alternatively through the nose NS) and then further advanced through the trachea TR to place the catheter 10 adjacent to a target pulmonary tissue in the trachea TR. The expandable member of the delivery catheter 10 may be expanded to advance a needle through a wall of the trachea to deliver a diagnostic and/or therapeutic agent to a target site in the trachea, for example, sub-epithelial tissue in the tissue such as submucosal tissue, smooth muscle tissue, the lamina propria, and the adventitia, to name a few targets. As shown in FIG. 15A, in some embodiments, a tracheoscope 1510 may be used to aid in placement and guidance of catheter 10. In some embodiments, a guidewire may be used to aid in placement and guidance of catheter 10. Further, in some embodiments, a tracheoscope 1510 or guidewire may be used separately or in combination to aid in placement and guidance of catheter 10.

Similarly, and as shown in FIG. 15B, for transbronchial administration, a delivery catheter of any of the embodiments disclosed herein may be advanced through the mouth MT (or alternatively through the nose NS) of a patient and then further advanced through the trachea TR to place the catheter 10 adjacent to a target pulmonary tissue in the bronchus. The catheter 10 may also be advanced further past the trachea TR and into either of the left main bronchus LMB or right main bronchus RMB for delivery of pharmaceutical agent into pulmonary tissue of the left lung LL or right lung RL. The catheter 10 may also be advanced further past the left main bronchus LMB or right main bronchus RMB and into any downstream bronchial tube BT to place the catheter adjacent target pulmonary tissue of the left or right lung. As shown in FIG. 15B, in some embodiments, a bronchoscope 1520 may be used to aid in placement and guidance of catheter 10. In some embodiments, a guidewire may be used to aid in placement and guidance of catheter 10. Further, in some embodiments, a bronchoscope 1520 or guidewire may be used separately or in combination to aid in placement and guidance of catheter 10. The expandable member of the delivery catheter 10 may be expanded to advance a needle through a wall of the trachea to deliver a diagnostic and/or therapeutic agent to a target site in the bronchus (e.g., left main bronchus LMB, right main bronchus RMB, or any bronchial tube BT), for example, sub-epithelial tissue in the tissue such as submucosal tissue, smooth muscle tissue, the lamina propria, and the adventitia, to name a few targets.

In some embodiments, the pharmaceutical agent can be delivered by transvascular, transtracheal, or transbronchial injection(s) as described herein prior to, during or after bronchial thermoplasty.

Other drug delivery devices and methods which may be used for the treatment of pulmonary diseases as described herein include those described in U.S. Pat. Nos. 7,070,606, 7,141,041, 7,465,298, 7,691,080, 7,744,584, 8,016,786, 8,708,995, 8,721,500, 9,061,098, and 9,149,497, and U.S. patent application Ser. Nos. 14/063,604, 14/605,865, and 14/838,531, the contents of which are incorporated by reference.

### Experimental

Four porcine subjects were treated with two different drugs to test pharmacokinetics and toxicity in porcine bronchi. Dexamethasone was administered to 2 animals at low (1 mg/mL), medium (2 mg/mL), and high (4 mg/mL) doses. The other drug, α-1-antitrypsin (Alpha-1-antitrypsin or A1AT), was administered to the remaining animals at low (2 mg/mL), medium (10 mg/mL), and high (50 mg/mL) doses. The drugs were administered via injections in all lobes of the lungs at the varying dosages.

All animals survived until a scheduled termination and were sacrificed on day 30. All four animals had limited necropsies completed. Histopathological evaluation showed the following results:

Dexamethasone Treated Group.

This group was comprised of 2 animals. Both animals received low dose treatment in the right upper and middle lung lobes, and mid dose treatment in the right lower lung lobe. One subject received high dose treatment in the left upper lung lobe. Another subject received high dose treatment in the left upper and lower lung lobes.

Examination of the lung tissues in the group which received Dexamethasone showed a normal lung architecture, thin interalveolar septa, folded columnar epithelial cells of bronchiole, clearly seen alveolar sacs, normal pulmonary vessels, and normal fibrous tissues distribution. There were no abnormal findings noted in bronchial or bronchiolar walls. There were minimal multifocal inflammatory cell infiltrates present in the majority of sections examined. These infiltrates were predominantly comprised of lymphocytes and macrophages, and were considered to be a background change in this animal model.

A1AT Treated Group.

This group was comprised of 2 animals. Both animals received low dose treatment in the right upper and middle lung lobes, and mid dose treatment in the right lower lung lobe. One subject received high dose treatment in the left lower lung lobe, and the other received high dose treatment in the left upper lung lobe.

Examination of the lung tissues in the group which received A1AT showed a normal lung architecture, thin interalveolar septa, columnar epithelial cells of bronchiole, clearly seen alveolar sacs, normal pulmonary vessels, and normal fibrous tissues distribution. There were no abnormal findings noted in bronchial or bronchiolar walls. There were minimal multifocal inflammatory cell infiltrates present in the majority of sections examined. These infiltrates were predominantly comprised of lymphocytes and macrophages, and were considered to be a background change in this animal model. There were no other abnormal findings present in this group.

The presence on multifocal inflammatory cell infiltrates was more likely a background change that can be seen in this animal model. Infiltrates were small, not associated with other findings such as fibrosis or necrosis, and were present in both treatment groups. They were not considered to be clinical significant.

In conclusion, the injections of Dexamethasone or A1AT did not induce any gross or histopathological changes in the lung that can be related to their toxic effects. There were normal lung architecture, thin interalveolar septa, columnar epithelial cells of bronchiole, clearly seen alveolar sacs, normal pulmonary vessels, and normal fibrous tissues distribution. There were no abnormal findings noted in bronchial or bronchiolar walls. Taken together these observations suggest the safety/absence of toxicity of the tested substances used in this animal model at the ˜30 day time point. This experiment illustrated that Dexamethasone or A1AT could be delivered in humans to accomplish the goals stated in this application, to relieve inflammation or to correct asthmatic conditions.

In another animal study, one porcine subject was utilized. The animal received multiple injections in the bronchial wall of sub-selected bronchi. First injections of methacholine in concentrations of 0.3 or 3.0 mg/mL and volumes of 0.1 to 0.5 mL were made into bronchial walls throughout the airway tree. Each injection resulted in immediate bronchoconstriction which lasted for more than 30 minutes. Next, injections of 0.1 to 0.5 mL of 4.0 to 400 μg/mL levalbuterol or 0.5 to 5 μg/mL of tiotropium bromide were injected into the narrowed segments, which resulted in immediate bronchodilation to relieve the bronchoconstriction. This demonstration showed that drugs could be locally and focally delivered to challenge or relieve the airway, illustrating that in human disease, the drugs could be focused on specific areas to relieve airway constriction, allowing treatment of asthma (using the bronchodilator) or diagnosis of hyperconstrictive areas of the airway (using the bronchoconstrictor).

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

What is claimed is:

1. A method for inhibiting an inflammatory pulmonary disease in a patient, the method comprising:
advancing a delivery catheter through a bodily lumen of a patient to a position adjacent a target site in pulmonary tissue;
advancing a delivery needle laterally from a lateral side of the delivery catheter through a wall of the bodily lumen to access the target site; and
injecting a therapeutically effective dose of a pharmaceutical agent to the target site.

2. The method of claim 1, wherein the therapeutically effective dose of the pharmaceutical agent is effective to suppress or prevent initiation, progression, or relapses of disease, including the progression of established disease.

3. The method of claim 1, wherein advancing the delivery catheter through the bodily lumen comprises advancing the delivery catheter through a blood vessel, and wherein advancing the delivery needle laterally from the lateral side of the delivery catheter through the wall of the bodily lumen comprises advancing the delivery needle through a wall of the blood vessel.

4. The method of claim 1, wherein advancing the delivery catheter through the bodily lumen comprises advancing the delivery catheter through a trachea, and wherein advancing the delivery needle laterally from the delivery catheter through the wall of the bodily lumen comprises advancing the delivery needle through a wall of the trachea.

5. The method of claim 1, wherein advancing the delivery catheter through the bodily lumen comprises advancing the delivery catheter through a bronchus or bronchi, and wherein advancing the delivery needle laterally from the delivery catheter through the wall of the bodily lumen comprises advancing the delivery needle through a wall of the bronchus or bronchi.

6. The method of claim 1, wherein advancing the delivery needle laterally from the lateral side of the delivery catheter comprises expanding an expandable element disposed on a distal portion of the catheter to extend the delivery needle laterally from the expandable element, thereby placing a section of the expandable element adjacent the delivery needle in contact with the wall of the bodily lumen.

7. The method of claim 6, wherein the section of the expandable element adjacent the delivery needle in contact with the wall of the bodily lumen seals and prevents leakage of the pharmaceutical agent delivered from the laterally extended delivery needle back into the bodily lumen.

8. The method of claim 6, wherein the section of the expandable element adjacent the delivery needle in contact with the wall of the bodily lumen seals a tissue tract of the laterally extended delivery needle.

9. The method of claim 1, wherein the inflammatory pulmonary disease comprises asthma, COPD, or infection.

10. The method of claim 1, further comprising diagnosing the patient as having the inflammatory pulmonary disease prior to injecting the therapeutically effective dose of the pharmaceutical agent.

11. The method of claim 1, further comprising monitoring the status of the patient affected by the pulmonary inflammatory disease following injecting the therapeutically effective dose of the pharmaceutical agent.

12. The method of claim 11, wherein the pulmonary tissues are monitored by MRI, x-ray, CT, spirometry, PCR, ELISA, NGS, or culture.

13. The method of claim 1, wherein the pharmaceutical agent comprises one or more of an antibiotic, DMARD, steroid, NSAID, smooth muscle relaxant, EGFR antagonist, PDGFR antagonist, PI3K inhibitor, neurotransmitter receptor inhibitor, growth factor receptor inhibitor, or protease inhibitor.

14. The method of claim 1, wherein the pharmaceutical agent is administered in combination with one or more pharmaceutical agent.

15. The method of claim 1, wherein the pharmaceutical agent comprises one or more of alpha-1-antitrypsin, tofacitinib, scopolamine, ceftriaxone, anti-IL5 antibody, anti-IL13 antibody, anti-33 antibody, prednisolone, or dexamethasone.

16. The method of claim 1, wherein the therapeutically effective dose of the pharmaceutical agent is injected prior to, during, or following bronchial thermoplasty.

17. The method of claim 1, wherein the pharmaceutical agent comprises one or more of albuterol, levalbuterol or pirbuterol.

18. The method of claim 1, wherein the pharmaceutical agent comprises one or more of tiotropium bromide, theophylline, hydralazine, clenbuterol, flavoxate, dicycloverine, papaverine, hyoscine hydrobromide, carisoprodol, cyclobenzaprine, metataxalone, methocarbamol, tizanidine, diazepam, baclofen, a substance P inhibitor, dantrolene, chlorzoxazone, gabapentin, or orphenadrine.

19. A pharmaceutical agent for use in a method of inhibiting an inflammatory pulmonary disease, wherein said pharmaceutical agent is for delivery to a target site in pulmonary tissue by micro-needle catheter, bypassing the pulmonary mucosal epithelial layer.

20. The pharmaceutical agent for use according to claim 19, wherein the pharmaceutical agent suppresses or prevents initiation, progression, or relapses of the disease, including the progression of established disease.

21. The pharmaceutical agent for use according to claim 19, wherein the pharmaceutical agent is for delivery by a pre-situated micro-needle catheter that has previously been advanced through a bodily lumen to a position adjacent to the target site, and wherein the micro-needle for delivery is extended laterally from a lateral side of the catheter through a wall of the bodily lumen to access the target site prior to the delivery of the pharmaceutical agent.

22. The pharmaceutical agent for use according claim 21, wherein:
(a) the bodily lumen is a blood vessel;
(b) the bodily lumen is a trachea; or
(c) the bodily lumen is a bronchus.

23. The pharmaceutical agent for use according to claim 21, wherein:
(a) the bodily lumen is a blood vessel and the micro-needle for delivery is extended laterally from the lateral side of the catheter through a wall of the blood vessel to access the target site;
(b) the bodily lumen is a trachea and the micro-needle for delivery is extended laterally from the lateral side of the catheter through a wall of the trachea to access the target site; or
(c) the bodily lumen is a bronchus and the micro-needle for delivery is extended laterally from the lateral side of the catheter through a wall of the bronchus to access the target site.

24. The pharmaceutical agent for use according to claim 19, wherein extending the micro-needle laterally from the lateral side of the catheter prior to delivery of the pharmaceutical agent comprises expanding an expandable element disposed on a distal end of the catheter to extend the needle laterally from the expandable element, thereby placing a section of the expandable element adjacent the needle in contact with a wall of the lumen.

25. The pharmaceutical agent for use according to claim 24, wherein:
(a) the section of the expandable element adjacent the needle in contact with the wall of the lumen prevents leakage of the pharmaceutical agent from the laterally extended needle back into the lumen; and/or
(b) extension of the needle through the wall of the bodily lumen generates a tissue tract, and wherein the section of the expandable element adjacent to the needle in contact with the wall of the lumen seals the tissue tract from the bodily lumen.

26. The pharmaceutical agent for use according to claim 19, wherein the inflammatory pulmonary disease is asthma, COPD or infection.

27. The pharmaceutical agent for use according to claim 19, wherein a patient to be treated is diagnosed as having the inflammatory pulmonary disease prior to delivery of the pharmaceutical agent.

28. The pharmaceutical agent for use according to claim 19, wherein the status of a patient affected by the pulmonary inflammatory disease is monitored following delivery of the pharmaceutical agent.

29. The pharmaceutical agent for use according to claim 19, wherein pulmonary tissues of the patient are monitored by MRI, x-ray, CT, spirometry, PCR, ELISA, NGS, or culture.

30. The pharmaceutical agent for use according to claim 19, wherein the pharmaceutical agent comprises one or more of an antibiotic, DMARD, steroid, NSAID, smooth muscle relaxant, EGFR antagonist, PDGFR antagonist, PI3K inhibitor, neurotransmitter receptor inhibitor, growth factor receptor inhibitor, or protease inhibitor.

31. The pharmaceutical agent for use according to claim 19, wherein the pharmaceutical agent is administered in combination with one or more additional pharmaceutical agent.

32. The pharmaceutical agent for use according to claim 19, wherein the pharmaceutical agent comprises one or more of alpha-1-antitrypsin, tofacitinib, scopolamine, ceftriaxone, anti-IL5 antibody, anti-IL13 antibody, anti-33 antibody, prednisolone, or dexamethasone.

33. The pharmaceutical agent for use according to claim 19, wherein the pharmaceutical agent is for delivery prior to, during, or following bronchial thermoplasty.

34. The pharmaceutical agent for use according to claim 19, wherein the pharmaceutical agent comprises one or more of albuterol, levalbuterol or pirbuterol.

35. The pharmaceutical agent for use according to claim 19, wherein the pharmaceutical agent comprises one or more of tiotropium bromide, theophylline, hydralazine, clenbuterol, flavoxate, dicycloverine, papaverine, hyoscine hydrobromide, carisoprodol, cyclobenzaprine, metataxalone, methocarbamol, tizanidine, diazepam, baclofen, a substance P inhibitor, dantrolene, chlorzoxazone, gabapentin, or orphenadrine.
